(19) United States
(12) Patent Application Publication
(10) Pub. No.: US 2020/0375904 A1
(43) Pub. Date: Dec. 3, 2020

(54) METHOD FOR MANUFACTURING DRUG-CONTAINING PARTICLES

(71) Applicant: Sumitomo Dainippon Pharma Co., Ltd., Osaka-shi (JP)

(72) Inventors: Manami OKA, Narita-shi (JP); Masaru YOSHIDA, Suita-shi (JP)

(73) Assignee: Sumitomo Dainippon Pharma Co., Ltd., Osaka-shi (JP)

(21) Appl. No.: 16/970,150

(22) PCT Filed: Feb. 15, 2019
(86) PCT No.: PCT/JP2019/005704

§ 371 Date: Aug. 14, 2020

(30) Foreign Application Priority Data
Feb. 16, 2018 (JP) 2018-026095

(51) Int. Cl. A61K 9/16; A61K 9/20; A61K 47/38; A61K 47/32; A61J 3/02

(52) U.S. Cl. CPC A61K 9/1682; A61K 9/2095; A61J 3/02; A61K 47/32; A61K 47/38

(57) ABSTRACT

The present invention relates to an efficient production method of a medicament-containing particle. According to the present invention, it is possible to provide a method for producing a hollow particle composed of a shell and a hollow, wherein the shell contains a medicament and a polymer, the method including a step of adding a polymer and a solvent capable of dissolving the polymer to a powder containing a medicament while rotating a container and a stirring blade by using a rotating mixing pan, and granulating the mixture by rotating the container and the stirring blade of the rotating mixing pan, wherein the polymer used as a starting material has an average particle size of not less than 5-fold that of the medicament used as a starting material.

### TECHNICAL FIELD

The present invention relates to a method for producing a medicament-containing particle which contains a medicament, specifically, a production method of a hollow particle containing a medicament as a main component in a wall (shell) part.

### BACKGROUND ART

In solid pharmaceutical preparations, in general, a medicament alone is granulated, or a medicament and other formulated component are mixed and granulated to produce medicament-containing particles, which are then mixed with other components, mixed with other granules, or added with other components, further granulated and the like, and the mixture is tableted to give tablets, or formulated to give granules, or packed in a capsule to give a capsule agent.

Patent document 1 discloses that a medicament-containing particle having a sufficient particle strength, and capable of exerting polymer function such as good disintegration property of a particle itself, dissolution control at a desired site and the like can be produced efficiently by a highly convenient means including mixing a powder mixture of a medicament and a polymer, particularly a polymer having desired functionality, and granulating by stirring the mixture while spraying a solvent capable of dissolving the polymer can be produced efficiently. Patent document 1 also discloses that the medicament-containing particle is spherical, has a hollow structure, the particle size and the particle size distribution width of the medicament-containing particle can be freely controlled, the particle has good flowability, its medicament content can be increased, and that the particle has superior particle homogeneity and shows good mixing uniformity with other components.

### DOCUMENT LIST

### Patent Document

patent document 1: WO 2014/030656

### SUMMARY OF INVENTION

### Technical Problem

The medicament-containing particle described in patent document 1 can be produced by a convenient method including an agitating granulation while spraying a solvent capable of dissolving a polymer. However, in the step of spraying the solvent, setting the conditions for liquid feeding rate, control of spray air and the like is sometimes difficult.

### Solution to Problem

The present inventors have conducted intensive studies and found that medicament-containing particles can be produced without spraying a solvent but by adding appropriate amounts of a polymer and a solvent capable of dissolving the polymer to a powder containing a medicament while rotating a container and a stirring blade by using a rotating mixing pan, and stirring the mixture by rotating the container and the stirring blade. In addition, the present inventors have confirmed that the medicament-containing particles produced by the method are spherical and have a hollow structure.

Furthermore, the present inventors have confirmed that the use of a rotating mixing pan equipped with a scraper does not require setting of complicated conditions or stopping the device found in conventional agitating granulators, decreases adhesion to side walls during production, and remarkably improves recovery efficiency of medicament-containing particles in the production step, irrespective of whether the solvent is added by dropwise addition or spraying.

That is, the present invention relates to the following.
[Item 1] A method for producing a medicament-containing particle composed of a shell and a hollow, wherein the shell comprises a medicament and a polymer, the method comprising a step of adding a polymer and a solvent capable of dissolving the polymer to a powder comprising a medicament while rotating a container and a stirring blade by using a rotating mixing pan, and granulating the mixture by rotating the container and the stirring blade of the rotating mixing pan, wherein the polymer used as a starting material has an average particle size of not less than 5-fold that of the medicament used as a starting material (when a polymer in a dispersion state is used, the polymer is powderized and then used as a powder).
[Item 2] The production method of Item 1, wherein the polymer used as a starting material has an average particle size of not less than 10-fold that of the medicament used as a starting material.
[Item 3] A method for producing a medicament-containing particle composed of a shell and a hollow, wherein the shell comprises a medicament and a polymer, the method comprising a step of adding a polymer and a solvent capable of dissolving the polymer to a powder comprising a medicament while rotating a container and a stirring blade by using a rotating mixing pan, and granulating the mixture by rotating the container and the stirring blade of the rotating mixing pan, wherein a particle size distribution ratio (D10/D90) of the polymer and the medicament is one or more (when a polymer in a dispersion state is used, the polymer is powderized and then used as a powder).
[Item 4] A method for producing a medicament-containing particle composed of a shell and a hollow, wherein the shell comprises a medicament, a polymer and other additive, the method comprising a step of adding a polymer and a solvent capable of dissolving the polymer to a powder mixture comprising a medicament and other additive while rotating a container and a stirring blade by using a rotating mixing pan, and granulating the mixture by rotating the container and the stirring blade of the rotating mixing pan, wherein the polymer used as a starting material has an average particle size of not less than 5-fold that of a mixed powder of the medicament used as a starting material and other additive (when a polymer in a dispersion state is used, the polymer is powderized and then used as a powder).
[Item 5] The production method of Item 4, wherein the polymer used as a starting material has an average particle size of not less than 10-fold that of the mixed powder of the medicament used as a starting material and other additive.
[Item 6] A method for producing a medicament-containing particle composed of a shell and a hollow, wherein the shell comprises a medicament, a polymer and other additive, the method comprising a step of adding a polymer and a solvent capable of dissolving the polymer to a powder mixture comprising a medicament and other additive while rotating a container and a stirring blade by using a rotating mixing pan, and granulating the mixture by rotating the container and the stirring blade of the rotating mixing pan, wherein a particle size distribution ratio (D10/D90) of the polymer used as a starting material and the mixed powder of a medicament used as a starting material and other additive is one or more (when a polymer in a dispersion state is used, the polymer is powderized and then used as a powder).
[Item 7] The production method of any one of Items 1 to 6, wherein the rotating mixing pan has a scraper.
[Item 8] The production method of Item 7, wherein the polymer and the solvent capable of dissolving the polymer are added dropwise or added by spraying.
[Item 9] The production method of Item 8, wherein the polymer and the solvent capable of dissolving the polymer are added dropwise.
[Item 10] The production method of Item 8, wherein the polymer and the solvent capable of dissolving the polymer are added by spraying.
[Item 11] The production method of any one of Items 1 to 10, wherein a volume ratio of the hollow relative to the whole particle of the medicament-containing particle is 1%-50%, and a shell thickness is not less than 15 μm.
[Item 12] The production method of any one of Items 1 to 11, wherein the polymer is one or more kinds selected from the group consisting of a water-soluble polymer, a water-insoluble polymer, an enteric polymer, a gastric soluble polymer and a biodegradable polymer.
[Item 13] The production method of Item 12, wherein the water-soluble polymer is selected from the group consisting of methylcellulose, hydroxypropylcellulose, hydroxypropylmethylcellulose, hydroxyethylcellulose, hydroxymethylcellulose, carboxymethylcellulose, polyvinylpyrrolidone, polyvinyl alcohol, copolyvidone, polyethylene glycol, polyvinyl alcohol-acrylic acid-methyl methacrylate copolymer, vinyl acetate-vinylpyrrolidone copolymer, polyvinyl alcohol-polyethylene glycol-graft copolymer, pregelatinized starch, dextrin, dextran, pullulan, alginic acid, gelatin, pectin, and a mixture of one or more kinds thereof.
[Item 14] The production method of Item 12, wherein the water-insoluble polymer is selected from the group consisting of ethylcellulose, acetyl cellulose, aminoalkylmethacrylate copolymer RS, ethyl acrylate-methyl methacrylate copolymer dispersion, vinyl acetate resin, and a mixture of one or more kinds thereof.
[Item 15] The production method of Item 12, wherein the enteric polymer is selected from the group consisting of hydroxypropylmethylcellulose acetate succinate, hydroxypropylmethylcellulose phthalate, methacrylic acid copolymer L, methacrylic acid copolymer LD, dried methacrylic acid copolymer LD, methacrylic acid copolymer S, methacrylic acid-n-butyl acrylate copolymer, and a mixture of one or more kinds thereof.
[Item 16] The production method of any one of Items 4 to 15, wherein said other additive is selected from the group consisting of filler, binder, sweetening agent, corrigent, smell masking agent, flavor, fluidizer, antistatic agent, colorant, disintegrant, lubricant, plasticizer, anticoagulant and coating agent.
[Item 17] The production method of any one of Items 1 to 16, wherein a rotating speed of the container is 0.1-1 m/s, and a rotating speed of the stirring blade is 5.9-34 m/s.

### Advantageous Effects of Invention

Using the production method of the present invention, medicament-containing particles can be produced in a shorter time than when the production method described in patent document 1 which includes an agitating granulation while spraying a solvent capable of dissolving a polymer is used, and more conveniently since control of the liquid feeding rate during spraying is not necessary, which in turn makes it possible to shorten the production time.

Furthermore, the production method using a rotating mixing pan equipped with a scraper of the present invention makes it possible to efficiently produce medicament-containing particles with less adhesion of the particles to the inner side walls of the device and without requiring control of the liquid feeding rate during conventional spraying, or the like, by any solvent addition method of dropwise or spraying.

### BRIEF DESCRIPTION OF DRAWINGS

FIG. 1 is an electron micrograph showing the appearance in Example 1.

FIG. 2 is an electron micrograph showing the cross-section in Example 1.

FIG. 3 is an electron micrograph showing the appearance in Example 2.

FIG. 4 is an electron micrograph showing the cross-section in Example 2.

FIG. 5 is a micro-CT scanner image of Example 1.

FIG. 6 is a micro-CT scanner image of Example 2.

FIG. 7 is an electron micrograph showing the appearance in Example 3.

FIG. 8 is an electron micrograph showing the appearance in Example 4.

FIG. 9 is an electron micrograph showing the appearance in Example 5.

FIG. 10 is an electron micrograph showing the appearance in Example 6.

FIG. 11 is an electron micrograph showing the appearance in Example 7.

FIG. 12 is an electron micrograph showing the appearance in Example 8.

FIG. 13 is an electron micrograph showing the appearance in Example 9.

FIG. 14 is an electron micrograph showing the appearance in Example 10.

FIG. 15 is an electron micrograph showing the appearance in Example 11.

FIG. 16 is an electron micrograph showing the appearance in Example 12.

FIG. 17 is an electron micrograph showing the appearance in Example 13.

FIG. 18 is an electron micrograph showing the appearance in Example 14.

FIG. 19 is an electron micrograph showing the appearance in Comparative Example 1.

FIG. 20 is an electron micrograph showing the appearance in Comparative Example 2.

FIG. 21 is an electron micrograph showing the appearance in Comparative Example 3.

FIG. 22 is an electron micrograph showing the appearance in Comparative Example 4.

FIG. 23 is a micro-CT scanner image of Example 3.

FIG. 24 is a micro-CT scanner image of Example 4.

FIG. 25 is a micro-CT scanner image of Example 5.

FIG. 26 is a micro-CT scanner image of Example 6.

FIG. 27 is a micro-CT scanner image of Example 7.

FIG. 28 is a micro-CT scanner image of Example 8.

FIG. 29 is a micro-CT scanner image of Example 9.

FIG. 30 is a micro-CT scanner image of Example 10.

FIG. 31 is a micro-CT scanner image of Example 11.

FIG. 32 is a micro-CT scanner image of Example 12.

FIG. 33 is a micro-CT scanner image of Example 13.

FIG. 34 is a micro-CT scanner image of Example 14.

FIG. 35 is a micro-CT scanner image of Comparative Example 1.

FIG. 36 is a micro-CT scanner image of Comparative Example 2.

FIG. 37 is a micro-CT scanner image of Comparative Example 3.

FIG. 38 is a micro-CT scanner image of Comparative Example 4.

### DESCRIPTION OF EMBODIMENTS

The present invention is explained in more detail in the is following.

The medicament-containing particle produced by the production method of the present invention contains a medicament and a polymer as essential constituent elements. The particle means both one particle and an aggregate of a plurality of particles.

In the present specification, the “average particle size” means cumulative 50% particle size D50 in the volume based measurement of powder particles. Such average particle size is measured by a laser diffraction particle size analyzer (e.g., Particle Viewer (manufactured by POWREX CORPORATION), or SALD-3000J (manufactured by Shimadzu Corporation), or HELOS&RODOS (manufactured by Sympatec GmbH)) by volume basis.

### (i) Medicament

Medicaments can be used without a particular limitation. The “medicament” to be used for the method of the present invention may be any medicament or compound irrespective of properties such as basic, acidic, ampholytic, neutral and the like, and solubility. Among those, from the aspects of stability and easy handling, a crystalline medicament or compound is preferable. In addition, a mixture of one or more kinds of medicaments may be used. The particle of the present invention is also effective for medicaments having low solubility. Far example, when the following water-soluble polymer is used as the polymer, rapid disintegration property and/or rapid dissolution property can be exhibited.

A smaller average particle size of a medicament used as a starting material in the present invention can afford a medicament-containing particle having a smoother surface. The average particle size of a medicament is preferably not more than 20 μm, more preferably not more than 10 μm, further preferably not more than 5 μm, most preferably not more than 3 μm. The average particle size of a medicament is generally not less than 0.1 μm.

The average particle size of a medicament used in the present invention may be any as long as it is within the above-mentioned range as a starting material, and may vary depending on the production process of the medicament-containing particle and the like.

Where necessary, the medicament may be pulverized to have a desired particle size before production of particles. While the pulverization is performed by a conventional method such as pulverization using a fine grinding mill and the like, very fine particles (average particle size not more than 1 μm) may be produced. While the medicament content can be set freely, the amount of the medicament to be used is generally not more than 96 wt %, preferably not more than 94 wt %, more preferably not more than 92 wt %, further preferably not more than 90 wt %, per 100 wt % of the medicament-containing particles (hollow particles) to be prepared. Specifically, it is 0.1-96 wt %, preferably 0.1-95.9 wt %, more preferably 1-94 wt %, further preferably 5-92 wt %, most preferably 10-90 wt %, per 100 wt % of the medicament-containing particles.

In the present invention, it is also possible to produce not only particles containing a medicament at a low content but also at a high content (e.g., 50-96 wt %, preferably 70-96 wt %, more preferably 90-96 wt %, per 100 wt % of the medicament-containing particles). When contained at a low content, the particles can be produced by mixing other additives, preferably, additives insoluble in solvents, which are described below.

### (ii) Polymer

In the present specification, the “polymer” refers to a molecule having a large relative molecular mass, and a structure composed of multiple repeats of a molecular having a small relative molecule mass, and particularly refers to a functional polymer. The aforementioned “molecule having a large relative molecular mass” has an average molecular weight (weight average molecular weight) of generally not less than 1000, preferably not less than 5000, more preferably not less than 10000. While the upper limit of the molecular weight is not particularly defined, it is preferably not more than 10000000, more preferably not more than 5000000, further preferably not more than 2000000, particularly preferably not more than 1000000. Examples of the functional polymer include water-soluble polymer, water-insoluble polymer, enteric polymer, gastric soluble polymer, and biodegradable polymer used for colon-targeting such as chitosan and the like. Preferred are water-soluble polymer, water-insoluble polymer, enteric polymer, and gastric soluble polymer. A mixture of one or more kinds of polymers may be used.

Examples of the water-soluble polymer include cellulose derivatives such as methylcellulose (e.g., trade name: SM-4, SM-15, SM-25, SM-100, SM-400, SM-1500, SM-4000, 60SH-50, 60SH-4000, 60SH-10000, 65SH-50, 65SH-400, 65SH-4000, 90SH-100SR, 90SH-4000SR, 90SH-15000SR, 90SH-100000SR), hydroxypropylcellulose (e.g., trade name: HPC-SSL, HPC-SL, HPC-L, HPC-M, HPC-H), hydroxypropylmethylcellulose (e.g., trade name: TC5-E, TC5-M, TC5-R, TC5-S, SB-4), hydroxyethylcellulose (e.g., trade name: SP200, SP400, SP500, SP600, SP850, SP900, EP850, SE400, SE500, SE600, SE850, SE900, EE820), hydroxymethylcellulose, carboxymethylcellulose (e.g., trade name: NS-300) and the like, and salts thereof, water-soluble vinyl derivatives such as polyvinylpyrrolidone (e.g., trade name: Plasdone K12, Plasdone K17, Plasdone K25, Plasdone K29-32, Plasdone K90, Plasdone K900), polyvinyl alcohol (e.g., trade name: Gohsenol EG-05, Gohsencl EG-40, Gohsenol EG-05P, Gohsenol EG-05PW, Gohsenol EG-30P, Gohsenol EG-30PW, Gohsenol EG-40P, Gohsenol EG-40PW), Copolyvidone (e.g., trade name: Kollidon VA64, Plasdone S-630), polyethylene glycol, polyvinyl alcohol-acrylic acid-methyl methacrylate copolymer (e.g., trade name: POVACOAT), vinyl acetate-vinylpyrrolidone copolymer (e.g., trade name: Kollidon VA64), polyvinyl alcohol-polyethylene glycol-graft copolymer (e.g., trade name: Kollicoat IR) and the like, pregelatinized starch (e.g., trade name: AMICOL C), dextrin, dextran, pullulan, alginic acid, gelatin, pectin and the like. A mixture of one or more kinds of water-soluble polymers may be used. Preferred are hydroxypropylcellulose, hydroxypropylmethylcellulose, polyvinylpyrrolidone, polyvinyl alcohol and pregelatinized starch, and more preferred is hydroxypropylcellulose.

Examples of the water-insoluble polymer include water insoluble cellulose ether such as ethylcellulose (e.g., trade name: ETHOCEL (ETHOCEL 10P)), acetyl cellulose and the like, water insoluble acrylic acid copolymers such as aminoalkylmethacrylate copolymer RS (e.g., trade name: Eudragit RL100, Eudragit RLPO, Eudragit RL30D, Eudragit RS100, Eudragit RSPO, Eudragit RS30D), ethyl acrylate-methyl methacrylate copolymer dispersion (e.g., trade name: Eudragit NE30D) and the like, vinyl acetate resin and the like. A mixture of one or more kinds of water-insoluble polymers may be used. Preferred are ethylcellulose and aminoalkylmethacrylate copolymer RS. In the present invention, sustained-release and the function of a bitter taste masking for a medicament having a bitter taste can be imparted by using a water-insoluble polymer as the polymer.

Examples of the enteric polymer include hydroxypropylmethylcellulose acetate succinate (e.g., trade name: AQOAT LF, AQOAT MF, AQOAT HF, AQOAT LG, AQOAT MG, AQOAT HG), hydroxypropylmethylcellulose phthalate (e.g., trade name: HPMCP50, HPMCP55, HPMCP55S), methacrylic acid copolymers such as methacrylic acid copolymer L (e.g., trade name: Eudragit L100), methacrylic acid copolymer LD (e.g., trade name: Eudragit L30D-55), dried methacrylic acid copolymer LD (e.g., trade name: Eudragit L100-55), methacrylic acid copolymer S (e.g., trade name: Eudragit S100), methacrylic acid-n-butyl acrylate copolymer and the like, and the like. A mixture of one or more kinds of enteric polymers may be used. Preferred are methacrylic acid copolymer L, and dried methacrylic acid copolymer LD.

Examples of the gastric soluble polymer include gastric soluble polyvinyl derivatives such as polyvinyl acetal diethylaminoacetate and the like, gastric soluble acrylic acid copolymers such as aminoalkylmethacrylate copolymer E (e.g., trade name: Eudragit E100, Eudragit EPO) and the like, and the like. A mixture of one or more kinds of gastric soluble polymers may be used. Preferred is aminoalkylmethacrylate copolymer E.

Biodegradable polymer is a polymer decomposable in vivo. Examples thereof include polylactic acid, polyglycolic acid, polycaprolactone and copolymers thereof, collagen, chitin, chitosan (e.g., trade name: FLONAC C-100M) and the like. A mixture of one or more kinds of biodegradable polymers may be used. Preferred are polylactic acid, polyglycolic acid, polycaprolactone and copolymers thereof, gelatin, collagen, chitin, and chitosan.

In the present invention, polymer can be selected depending on the purpose. For example, to achieve rapid dissolution of medicament from a medicament particle in the gastrointestinal tract, a water-soluble polymer is preferably used as the polymer; to achieve sustained-release of a medicament, a water-insoluble polymer is preferably used as the polymer; to achieve a bitter taste masking, a water-insoluble polymer, an enteric polymer, a gastric soluble polymer and the like are preferably used; to suppress dissolution of medicament in the stomach, and facilitate dissolution in the small intestine, an enteric polymer is preferably used; and to achieve colon-targeting, chitosan and the like are preferably used. Depending on the purpose, a mixture of two or more kinds of polymers having different functions such as a water-soluble polymer, a water-insoluble polymer and the like may be used.

In the present invention, a polymer in a powdery condition is preferably used, and a polymer having a suitable average particle size and suitable particle size distribution can be selected depending on the desired average particle size and particle size distribution of the medicament-containing particle. While the examples of the polymer recited above also include polymers in a dispersion state, they can be used as a powder for the present invention by, for example, powderizing them by spray drying and the like. For example, to obtain a medicament-containing particle having a narrow particle size distribution, a polymer powder having a narrow particle size distribution is preferably used. To obtain a medicament-containing particle having a large average particle size, a polymer powder having a large average particle size is preferably used; and to obtain a medicament-containing particle having a small average particle size, a polymer powder having a small average particle size is preferably used. This in turn means that a medicament-containing particle having a particle size distribution suitable for the purpose can be produced by adjusting the size and particle size distribution of the polymer powder.

An average particle size of a polymer used as a starting material in the present invention is not less than 0.5 μm, preferably not less than 5 μm. In a preferred embodiment, the average particle size of a polymer is not less than 20 μm, not less than 25 μm, not less than 40 μm, not less than 50 μm. In addition, the average particle size of a polymer is generally not more than 5 mm, preferably not more than 1 mm, more preferably not more than 300 μm, further preferably not more than 250 μm, particularly preferably not more than 200 μm.

A polymer powder of a particular particle size fraction can also be used selectively by, for example, a sieving method. Specifically, a polymer having a desired particle size distribution can be obtained and used by appropriately selecting a sieve of a sieve number described in USP (United States Pharmacopeia), EP (European Pharmacopoeia), JP (the Japanese Pharmacopoeia), and fractionating a polymer powder. When the particle size distribution width (D90/D10) of a polymer used as a starting material is small, the particle size distribution width (D90/D10) of medicament-containing particle becomes small.

In the present invention, the average particle size of a polymer only needs to be within the above-mentioned range as a starting material, and may vary in the production process and the like of a medicament-containing particle.

In the present invention, the polymer is not used as a granulation liquid in a state of solution or suspension, but is mixed as a powder with a medicament and a solvent by using a rotating mixing pan, and the mixture is granulated by rotating the container and the stirring blade of the rotating mixing pan. As long as the effect of the invention can be exhibited, a part of the polymer or medicament may be used by dissolving or suspending in a solvent.

While the amount of the polymer to be used varies depending on the kind of the medicament, the amounts of other additive, particle size of the polymer, the strength of the binding force of the polymer and the like, it is generally used within the range of 4-50 wt %, preferably 4-40 wt %, more preferably 6-40 wt % or 8-40 wt %, further preferably 10-40 wt %, still more preferably 10-30 wt %, particularly preferably 10-20 wt %, per 100 wt % of the medicament-containing particles (hollow particles) to be produced.

In another embodiment, the amount of the polymer to be used is preferably 5-50 wt %, more preferably 5-40 wt %, further preferably 5-30 wt %, particularly preferably 5-25 wt %, per 100 wt % of the medicament-containing particles (hollow particles) to be produced.

For the production of the medicament-containing particle of the present invention, other additive may also be contained as necessary. The amount of other additive to be added can be appropriately adjusted according to the kind and amount of the medicament, polymer and/or solvent. Other additive can be added to the powder containing a medicament before addition of the polymer and solvent.

Other additive is not particularly limited as long as it is generally used and, for example, filler (e.g., starch such as rice starch and the like, D-mannitol, magnesium carbonate), binder, sweetening agent, corrigent, smell masking agent, flavor, fluidizer (e.g., aerosil), antistatic agent, colorant, disintegrant, lubricant, plasticizer, anticoagulant, coating agent and the like can be mentioned. The additive is not particularly limited and, when the corresponding polymer mentioned above does not dissolve in the solvent to be used, the polymer does not exhibit the function in the present invention and is added as an additive.

Other additive is preferably a powder. When other additive is a powder, an average particle size of the powder of other additive to be used as a starting material is not more than 20 μm, preferably not more than 10 μm, more preferably not more than 5 μm, further preferably not more than 3 μm. It is preferably the same as or smaller than the average particle size of the aforementioned medicament powder to be used as a starting material. When the average particle size of other additive is large, desired particle containing a polymer, a medicament, and other additive cannot be formed. When other additive is coarse, it is separated from the medicament-containing particle of the present invention. The amount of other additive to be used is not particularly limited, and a smaller amount thereof to be used affords particles with a high medicament content percentage. Particles with a small medicament content percentage can be produced by increasing the amount of the additive. It is also possible to add other additive dissolved or dispersed in a solvent. When other additive is dissolved, the average particle size thereof is not particularly limited. When other additive is dispersed, the average particle size thereof is preferably the same as or below the average particle size of a powder of the aforementioned additive. The average particle size of other additive is generally not less than 0.005 μm.

In the present invention, the average particle size of other additive as a starting material may be within the above-mentioned range, and may be changed in the production process and the like of the medicament-containing particle.

As the medicament-containing particle of the present invention, a particle containing 60-96 wt % of a medicament, and 4-40 wt % of a polymer, each per 100 wt % of the medicament-containing particle can be mentioned. Particularly preferably, a particle containing 70-95 wt % of a medicament, and 5-30 wt % of a polymer, more preferably, a particle containing 80-90 wt % of a medicament, and 10-20 wt % of a polymer can be mentioned.

As the medicament-containing particle containing other additive of the present invention, a particle containing 55 -95.9 wt % of a medicament, 4-40 wt % of a polymer, and 0.1-5 wt % of other additive, each per 100 wt % of the medicament-containing particle can be mentioned. Particularly preferably, a particle containing 65-94.9 wt % of a medicament, 5-30 wt % of a polymer, and 0.1-5 wt % of other additive, more preferably a particle containing 75-89.9 wt % of a medicament, 10-20 wt % of a polymer, and 0.1-5 wt % of other additive can be mentioned.

As the medicament-containing particle containing other additive of the present invention, a particle containing 0.1 -95.9 wt % of a medicament, 4-40 wt % of a polymer, and 0.1 -95.9 wt % of other additive, each per 100 wt % of the medicament-containing particle can be mentioned. Particularly preferably, a particle containing 1-94 wt % of a medicament, 5-30 wt % of a polymer, and 1-94 wt % cf other additive, more preferably a particle containing 10-80 wt % of a medicament, 10-20 wt % of a polymer, and 10-80 wt % of other additive can be mentioned.

The average particle size of the polymer used as a starting material is generally not less than 5-fold, preferably not less than 10-fold, more preferably not less than 15-fold, further preferably not less than 20-fold, particularly preferably not less than 25-fold, that of the medicament used as a starting material. The average particle size of the polymer used as a starting material is generally not more than 10000000-fold, preferably not more than 1000-fold, more preferably not more than 500-fold, further preferably not more than 100-fold, that of the medicament used as a starting material.

When other additive is used, the average particle size of a mixed powder of a medicament used as a starting material and other additive is important for the production of a medicament-containing particle. In this case, the average particle size of a polymer used as a starting material is generally not less than 5-fold, preferably not less than 10-fold, more preferably not less than 15-fold, particularly preferably not less than 25-fold, that of a mixed powder of a medicament used as a starting material and other additive. In addition, the average particle size of the polymer used as a starting material is generally not more than 10000000-fold, preferably not more than 1000-fold, more preferably not more than 500-fold, further preferably not more than 100-fold, that of a mixed powder of the medicament used as a starting material and other additive.

In the present specification, the “powder containing a medicament” may be a powdery form containing a medicament, and includes a powder containing only one kind of medicament, a powder mixture containing two or more kinds of different medicaments, and the like.

In the present specification, the number of folds of the average particle size of a polymer used as a starting material relative tc that of a medicament used as a starting material is shown by a particle size distribution ratio (D50/D50) of the average particle size of a polymer used as a starting material and that of a medicament used as a starting material. Similarly, the number of folds of the average particle size of a polymer used as a starting material relative to that of a mixed powder of a medicament used as a starting material and other additive is shown by a particle size distribution ratio (D50/D50) of the average particle size of a polymer used as a starting material and that of a mixed powder of a medicament used as a starting material and other additive.

It is preferable that the particle size distribution of a polymer used as a starting material should not overlap with the particle size distribution of a medicament used as a starting material. Specifically, for example, cumulative 10% particle size D10 of a polymer used as a starting material in a volume based measurement is preferably larger than cumulative 90% particle size D90 of a medicament used as a starting material. In other words, cumulative 10% particle size D10 of the polymer used as a starting material/cumulative 90% particle size D90 of a medicament used as a starting material [particle size distribution ratio (D10/D90) of polymer and medicament] is preferably not less than 1, more preferably not less than 2, further preferably not less than 4. In addition, the particle size distribution ratio (D10/D90) of polymer and medicament is generally not more than 5000000, preferably not more than 500, more preferably not more than 250, further preferably not more than 50.

When other additive is used, cumulative 10% particle size D10 of a polymer used as a starting material in a volume based measurement is preferably larger than cumulative 90% particle size D90 of a mixed powder of a medicament used as a starting material and other additive. In other words, cumulative 10% particle size D10 of the polymer used as a starting material/cumulative 90% particle size D90 of a mixed powder of a medicament used as a starting material and other additive [particle size distribution ratio (D10/D90) of a mixed powder of polymer, medicament, and other additive] is preferably not less than 1, more preferably not less than 2, further preferably not less than 4. In addition, the particle size distribution ratio (D10/D90) of a mixed powder of a polymer used as a starting material, a medicament used as a starting material, and other additive is generally not more than 5000000, preferably not more than 500, more preferably not more than 250, further preferably not more than 50.

The “solvent” in the present specification means any solvent acceptable in the fields of pharmaceutical product, quasi-medicament, cosmetic, food and the like, and may be any as long as it can dissolve a polymer to be used. Since the medicament-containing particle of the present invention is used as a medicament, a pharmaceutically acceptable solvent is preferable. Such solvent is appropriately selected depending on the kind of the medicament, polymer and additive, and the like, and a mixture of several kinds of solvents may be used.

Examples of the “solvent” in the present invention include water, alcohol solvents (e.g., optionally substituted lower alkanol such as methanol, ethanol, n-propylalcohol, iso-propylalcohol, 2-methoxyethanol, 2-ethoxyethanol and the like), ketone solvents (e.g., lower alkylketone such as acetone, methylethylketone and the like), ester solvents (e.g., lower alkyl ester of acetic acid such as ethyl acetate and the like) and a mixed solvent thereof.

Specifically, when a water-soluble polymer is used as the polymer in the present invention, a solvent capable of dissolving the polymer (e.g., water, water-containing alcohol solvent etc.) can be used as the solvent, and water or water-containing ethanol can be particularly preferably used. When a water-insoluble polymer is used as the polymer, a solvent capable of dissolving the polymer (e.g., alcohol solvent, ketone solvent, ester solvent etc.) can be used as the solvent, and solvents capable of dissolving polymers such as gastric soluble polymer, enteric polymer, chitosan and the like (e.g., alcoholic solvent, more specifically ethanol) can be used as the solvent.

While the amount of the solvent to be used in the present invention varies depending on the kind, amount and the like of the medicament and polymer, it is generally 5-60 parts by weight, preferably 10-40 parts by weight, more preferably 15-25 parts by weight, further preferably 18-22 parts by weight, per 100 parts by weight of the total amount of the components constituting the medicament-containing particle.

In the present specification, the “addition” means a method of adding a solvent by dropwise or spraying, which is used in the production step of pharmaceutical products or the like. Any solvent can be used as long as it is applicable, and it is appropriately selected according to the kind, properties and the like of the medicament, polymer, additive and the like contained in the medicament-containing particle of the present invention.

For the “addition” in the present invention, dropwise into a rotating mixing pan eliminates the need to control the liquid feeding rate during spraying and enables production in a short time. With a rotating mixing pan equipped with a scraper, adhesion of particles to the inner side of a device can be decreased and the recovery efficiency of medicament-containing particles from the device can be improved irrespective of the addition method of dropwise addition or spraying.

### Production Method of the Present Invention

The production method of the medicament-containing particle of the present invention is characterized in that a medicament-containing particle is obtained by adding a polymer and a solvent capable of dissolving the polymer to a powder containing a medicament (the above-mentioned (i)) while rotating a container and a stirring blade by using a rotating mixing pan, granulating the mixture by rotating the container and the stirring blade of the rotating mixing pan, and drying same.

The rotating speed of the container and the rotating speed of the stirring blade in the present invention are not particularly limited as long as the mixture flows. The rotating speed of the container is generally 0.1-1 m/s, preferably 0.4-1 m/s. The rotating speed of the stirring blade is generally 5.9-34 m/s, preferably 9.8-23 m/s. When the rotating speed is set tc RPM (Rotations Per Minute) depending on the device to be used, it can be calculated and set based on the following formula.

rotating speed (RPM)=rotating speed (m/s)×60/(diameter of container or stirring blade×circular constant (π))

Specifically, in the case of Intensive Mixer R02 (manufactured by NIPPON EIRICH CO., LTD.), since the diameter of the container is 0.235 m and the diameter of the stirring blade is 0.125 m, the range of the aforementioned rotating speed is as mentioned below. That is, the rotating speed of the container is generally 10-84 RPM, preferably 30-60 RPM. The rotating speed of the stirring blade is generally 900-5000 RPM, preferably 1500-3500 RPM.

In the case of Eirich CleanLine C5 (manufactured by NIPPON EIRICH CO., LTD.), since the diameter of the container is 0.235 m and the diameter of the stirring blade is 0.125 m, the range of the aforementioned rotating speed is as mentioned below. That is, the rotating speed of the container is generally 34-87 RPM, preferably 34-60 RPM. The rotating speed of the stirring blade is generally 360-5400 RPM, preferably 1500-3500 RPM.

In the case of Eirich CleanLine C50 (manufactured by NIPPON EIRICH CO., LTD.), since the diameter of the container is 0.600 m and the diameter of the stirring blade is 0.280 m, the range of the aforementioned rotating speed is as mentioned below. That is, the rotating speed of the container is generally 10-48.5 RPM, preferably 20-30 RPM. The rotating speed of the stirring blade is generally 500-2000 RPM, preferably 1000-2000 RPM.

While the method for adding the solvent in the present invention is not particularly limited, addition over 30 seconds or more is preferable since uniform addition to a powder or powder mixture is achieved. Specifically, injection or dropwise using a funnel, direct injection or dropwise from a container, and the like can be mentioned. In addition, a solvent may be sprayed in a conventional manner.

As for the order of “adding a polymer and a solvent capable of dissolving the polymer” in the present invention, either may be added first, and addition in any order may be included in the present invention.

The device to be used in the present invention may be any device as long as the container and the stirring blade rotate. For example, a rotating mixing pan, a rotary-type agitating mixer, a rotary-type forced agitating mixer and the like can be mentioned, and a preferred device is a rotating mixing pan. Specifically, for example, Intensive Mixer (manufactured by NIPPON EIRICH CO., LTD.), DMK-series mixer (manufactured by TIGER MACHINE Co. Ltd.) (manufactured by TIGER-CHIYODA MACHINERY CO. LTD.), MZ-series mixer (manufactured by MIG Co., Ltd.), Tsuvler (manufactured by NIKKO Co., Ltd.) and the like can be mentioned.

The device to be used in the present invention may be further provided with a scraper. The material of the scraper may be polytetrafluoroethylene, polyamide, SUS, and the like, and a preferred material is polyamide. In addition, to reduce the adhesion of the medicament-containing particles to the inner side wall of the rotating mixing pan, the scraper is provided at a distance of generally 0-50 mm, preferably 0 -10 mm, more preferably 0-3 mm, from the inner side wall of the rotating mixing pan.

As the drying method, a method known per se can be appropriately selected. For example, drying by shelf dryer or fluidized bed and the like can be mentioned and, from the aspects of productivity, drying by fluidized bed is preferable.

### Medicament-Containing Particle Produced by the Present Invention

The medicament-containing particle obtained by the production method of the present invention is a particle composed of a shell (or wall) and a hollow, wherein the shell contains a medicament and a polymer. Alternatively, it is a particle having a structure wherein a hollow is surrounded by a wall composed of a composition comprising a medicament and a polymer.

The aforementioned particle is characterized in that the particle has an inner hollow structure. The “hollow” here is different from the presence of many gaps at undetermined positions in general tablets, and refers to a completely independent single void present in the center of a particle, which is surrounded by the wall (shell) made of a medicament containing composition. For example, the presence thereof can be confirmed by an electron microscope or optical microscope.

The volume ratio of the hollow relative to the volume of the medicament-containing particle of the present invention as a whole is 1%-50%, preferably 1%-30%, more preferably 1.5%-30%, particularly preferably about 2%-30%. The aforementioned volume ratio of a hollow is determined by dividing the volume of the hollow by the volume of the particle. Since the particle produced by the production method of the present invention generally has high sphericity, the volume is determined by assuming that both the hollow and the particle are spheres. The volume of the hollow and the particle is calculated by determining the major diameter and the minor diameter of the hollow and the particle at the center of the particle by X ray CT (computerized tomography device) measurement, and determining the volume of the sphere assuming the average thereof to be hollow diameter and particle diameter.

To be specific, the “volume ratio of the hollow” in the present invention can be obtained by calculation by the following formula.

volume ratio of hollow [%]=(4/3×π×(diameter of hollow/2)³)/(4/3×π×(particle size of medicament-containing particle/2)³)×100

The particle size of the medicament-containing particle and the diameter of the hollow are non-destructively measured by a benchtop micro-CT (manufactured by SKYSCAN, SKYSCAN1172) and the average of 10 measurements is used.

The medicament-containing particle obtained by the production method of the present invention has a wall (shell) on the outside of the hollow. While the shell thickness of the medicament-containing particle of the present invention can be freely determined, the strength of the particle becomes low when the shell thickness is small. The shell thickness of the medicament-containing particle of the present invention is preferably not less than 10 μm, more preferably not less than 15 μm, further preferably not less than 20 μm, most preferably not less than 30 μm. The shell thickness can be measured by, for example, X-ray CT (computed tomography device).

The percentage of the shell thickness of the medicament-containing particle may be any in the present invention, and can be determined by the following formula. The percentage of the shell thickness is preferably 20-80%, more preferably 30-70%.

percentage of shell thickness [%]=(shell thickness/(particle size of medicament-containing particle/2))×100

The medicament-containing particle obtained by the production method of the present invention is characterized in that the particle size of the medicament-containing particle can be freely adjusted. Therefore, an average particle size of the medicament-containing particle can be adjusted to about 1 -7000 μm, preferably about 5-1000 μm, more preferably about 10-500 μm, further preferably about 10-400 μm, still more preferably about 20-300 μm, particularly preferably about 50-300 μm.

From the aspect of particle strength, the average particle size of the medicament-containing particle is preferably about 50-7000 μm, more preferably about 50-1000 μm, further preferably about 50-500 μm. In another embodiment, the average particle size of the medicament-containing particle can be adjusted to preferably about 70 -7000 μm, more preferably about 70-1000 μm, further preferably about 70-500 μm, particularly more preferably about 70-300 μm, most preferably about 100-300 μm.

In the present invention, the size of the obtained medicament-containing particle can be adjusted, as described above, by adjusting the average particle size of the polymer.

While the medicament-containing particle obtained by the production method of the present invention has a hollow, the diameter of the hollow is generally not less than 10 μm. In addition, the diameter of the hollow of the medicament-containing particle can be freely adjusted to generally about 10-5000 μm, preferably about 20-700 more preferably about 30-300 μm, further preferably about 50-200 μm. In the medicament-containing particle, the hollow ratio can be freely changed in association with the above-mentioned particle size (average particle size).

In the present specification, the “diameter of hollow” is determined by calculation by the following formula.

diameter of hollow [μm]=(major diameter of hollow+minor diameter of hollow)/2

The major diameter and minor diameter of the hollow of the particle are non-destructively measured by a benchtop micro-CT (manufactured by SKYSCAN, SKYSCAN1172) and the average of 10 measurements is used.

In the present specification, the “shell thickness” can be obtained by calculation by the following formula.

shell thickness [μm]=(particle size of medicament-containing particle−diameter of hollow)/2

The particle size of the medicament-containing particle, and the diameter of the hollow were non-destructively measured by a benchtop micro-CT (manufactured by SKYSCAN, SKYSCAN1172) and the average of 10 measurements was used.

In the present specification, the “percentage of the shell thickness” can be obtained by calculation by the following formula.

percentage of shell thickness [%]=(shell thickness/(particle size of medicament-containing particle/2))×100

The particle size of the medicament-containing particle is non-destructively measured by a benchtop micro-CT (manufactured by SKYSCAN, SKYSCAN1172) and the average of 10 measurements is used.

In the present specification, the “volume ratio of the hollow” can be obtained by calculation by the following formula.

volume ratio of hollow [%]=(4/3×π×(diameter of hollow/2)³)/(4/3×π×(particle size of medicament-containing particle/2)³)×100

The particle size of the medicament-containing particle and the diameter of the hollow are non-destructively measured by a benchtop micro-CT (manufactured by SKYSCAN, SKYSCAN1172) and the average of 10 measurements is used.

In one embodiment, the medicament-containing particle obtained by the production method of the present invention is spherical. As used herein, being “spherical” means having an aspect ratio of 1.0-1.5, preferably 1.0-1.4, more preferably 1.0-1.3. Having such shape, the medicament-containing particles show good fluidity when they are tableted, or filled in capsule and the like, and the efficiency is also improved during processing such as coating and the like.

In the present specification, the “aspect ratio” is a ratio of the minor diameter and the major diameter of a particle, and is an indication of the sphericity. The aspect ratio can be obtained by calculation by, for example, the following formula.

aspect ratio=major diameter of particle/minor diameter of particle

The major diameter and minor diameter of the particle are non-destructively measured by a benchtop micro-CT (manufactured by SKYSCAN, SKYSCAN1172), and the average of 10 measurements is used.

In addition, Millitrac JPA (NIKKISO CO., LTD.) may be used for the measurement.

In the present specification, the “particle size distribution ratio (D50/D50) of polymer and medicament” can be obtained by calculation by the following formula.

particle size distribution ratio of polymer and medicament (D50/050)=D50 of polymer/D50 of medicament

The “particle size distribution ratio (D50/D50) of polymer and mixed powder of medicament and other additive” in the present specification can be obtained by calculation by the following formula

particle size distribution ratio of polymer and mixed powder of medicament and other additive (D50/D50)=D50 of polymer/D50 of mixed powder of medicament and other additive

The particle size distribution of polymer, medicament, and mixed powder of medicament and other additive is measured by a laser diffraction particle size analyzer (e.g., Particle Viewer (manufactured by POWREX CORPORATION), SALD-3000) (manufactured by Shimadzu Corporation) or HELOS&RODOS (manufactured by Sympatec)) by volume basis.

In the present specification, the “particle size distribution ratio (D10/D90) of polymer and medicament” can be obtained by calculation by the following formula.

particle size distribution ratio of polymer and medicament (D10/D90)=D10 of polymer/D90 of medicament

The “particle size distribution ratio (D10/090) of polymer and mixed powder of medicament and other additive” in the present specification can be obtained by calculation by the following formula

particle size distribution ratio of polymer and mixed powder of medicament and other additive (D10/D90)=D10 of polymer/D90 of mixed powder of medicament and other additive

The particle size distribution of polymer, medicament, and mixed powder of medicament and other additive is measured by a laser diffraction particle size analyzer (manufactured by POWREX CORPORATION), SALD-3000J (manufactured by Shimadzu Corporation) or HELOS&RODOS (manufactured by Sympatec)) by volume basis.

In the present specification, “particle size distribution width is small” means that the specific particle size distribution ratio (e.g., D90/D10) is not more than 6.0.

The medicament-containing particle obtained by the production method of the present invention is useful as a medicament or a starting material of a medicament, and can be administered orally or parenterally to a human or animal. The dose can be appropriately selected according to the medicament to be used.

The medicament-containing particle obtained by the production method of the present invention is generally used as a medicament or pharmaceutical composition containing a plurality of the medicament-containing particle.

The medicament-containing particle obtained by the production method of the present invention can be formulated into various dosage forms according to the object of use thereof. For example, the medicament-containing particle can be used as it is, or granule, injection for preparation when in use, dosage form for implantation and the like. Moreover, the medicament-containing particle can also be mixed with any additive and tableted to give a tablet (including orally disintegrating tablet), or filled in a capsule to give a capsule agent. Furthermore, the medicament-containing particle can also be used as a suspension (aqueous suspension, oily suspension), emulsion and the like.

### EXAMPLE

The present invention is explained further specifically in the following by referring to Examples and Experimental Examples, which are not to be construed as limitative. Further, the present invention may be changed without departing from the scope of the present invention. The compound names shown in the following Examples and Experimental Examples do not always follow the IUPAC nomenclature.

In the Examples and Experimental Examples, unless particularly indicated, % of solvent means (W/W%) and % of particle means wt %.

Unless particularly indicated, the medicaments, polymers and additives used in the present Examples and Experimental Examples were the following.
5,5-diphenylimidazolidine-2,4-dione (phenytoin): MUROMACHI CHEMICALS INC benzo[d]isoxazol-3-ylmethanesulfonamide (zonisamide): JUZEN CHEMICAL CORPORATION N-(4-hydroxyphenyl)acetamide (acetaminophen): YAMAMOTO CORPORATION
lactose (SorboLac400): MEGGLE Japan CO., LTD. corn starch (cornstarch): Hinokuni food Corporation hydroxypropylcellulose (HPC-L): NIPPON SODA CO., LTD. ammonioalkylmethacrylate copolymer RS (Eudragit RSPO): EVONIK JAPAN CO., LTD.

The production instruments used for production in the present Examples are as follows.

In Examples 1, 2, Intensive Mixer R02 (manufactured by NIPPON EIRICH CO., LTD.) was used. The material of the scraper was polyamide, and the scraper was provided at a distance of 0-3 mm from the inner side wall of the rotating mixing pan.

In Examples 3-12, Eirich CleanLine C5 (manufactured by NIPPON EIRICH CO., LTD.) was used. The material of the scraper was polyamide, and the scraper was provided at a distance of 0-3 mm from the inner side wall of the rotating mixing pan.

In Examples 13-14, Eirich CleanLine C50 (manufactured by NIPPON EIRICH CO., LTD.) was used. The material of the scraper was polyamide, and the scraper was provided at a distance of 0-3 mm from the inner side wall of the rotating mixing pan.

The test methods in the present Examples and Experimental Examples were as follows.

### (Appearance and Cross-Section of Medicament-Containing Particle Obtained by the Production Method of the Present Invention)

The appearance and cross-section of the particles were observed under a scanning electron microscope (3D real surface view microscope, manufactured by KEYENCE CORPORATION, VE-8800). (Inner structure of medicament-containing particle)

The inner structure of the medicament-containing particle was non-destructively observed by a benchtop micro-CT (manufactured by SKYSCAN, SKYSCAN1172).

### Example 1

5,5-Diphenylimidazolidine-2,4-dione, lactose, and corn starch indicated in the following Table 1 were charged in a rotating mixing pan (Intensive Mixer R02, manufactured by NIPPON EIRICH CO., LTD.) and the mixture was premixed for 30 sec (stirring blade rotating speed: 1500 RPM (9.8 m/s), container rotating speed: 42 RPM (0.5 m/s)). Successively, with stirring (stirring blade rotating speed: 1500 RPM (9.8 m/s), container rotating speed: 42 RPM (0.5 m/s)), 205.34 g of water was added dropwise for 1 min using a funnel, and the mixture was stirred for 10 sec (stirring blade rotating speed: 3000 RPM (19.6 m/s), container rotating speed: 42 RPM (0.5 m/s)). Successively, with stirring (stirring blade rotating speed: 3000 RPM (19.6 m/s), container rotating speed: 42 RPM (0.5 m/s)), hydroxypropylcellulose indicated in Table 1 was charged for 50 sec, and the mixture was further stirred for 6 min 30 sec (stirring blade rotating speed: 3000 RPM (19.6 m/s), container rotating speed: 42 RPM (0.5 m/s)) to give medicament-containing particles in a wet powder state. The wet powdery particles were charged in a fluid bed granulator (MP-01, manufactured by POWREX CORPORATION) and dried to give the particles of Example 1. Since the mixed powder of 5,5-diphenylimidazolidine-2,4-dione, lactose, and corn starch showed D50 of 8.85 μm, D90 of 21.55 μm, and D50 of hydroxypropylcellulose was 142.37 μm and D10 was 47.18 μm, the “particle size distribution ratio of polymer, and mixed powder of medicament and other additive (D50/D50)” and the “particle size distribution ratio of polymer, and mixed powder of medicament and other additive (D10/D90)” calculated from the aforementioned calculation formulas were respectively 16 and 2.2.

TABLE 1

| | Example 1 |
|---|---|
| | charge |
| | amount |
| | (g) |
| 5,5-diphenylimidazolidine-2,4-dione | 300.00 |
| lactose | 455.00 |
| corn starch | 195.00 |
| hydroxypropylcellulose | 50.78 |
| water * | 205.34 |
| total | 1000.78 |

* not remaining in medicament-containing particles

### Example 2

5,5-Diphenylimidazolidine-2,4-dione, lactose, corn starch, and hydroxypropylcellulose indicated in the following Table 2 were charged in a rotating mixing pan (Intensive Mixer R02, manufactured by NIPPON EIRICH CO., LTD.) and the mixture was premixed for 30 sec (stirring blade rotating speed: 1500 RPM (9.8 m/s), container rotating speed: 42 RPM (0.5 m/s)). Successively, with stirring (stirring blade rotating speed: 1500 RPM (9.8 m/s), container rotating speed: 42 RPM (0.5 m/s)), 206.03 g of water was added dropwise for 1 min using a funnel, and the mixture was stirred for 5 min 30 sec (stirring blade rotating speed: 3000 RPM (19.6 m/s), container rotating speed: 42 RPM (0.5 m/s)) to give medicament-containing particles in a wet powder state. The wet powdery particles were charged in a fluid bed granulator (MP-01, manufactured by POWREX CORPORATION) and dried to give the particles of Example 2. The “particle size distribution ratio of polymer, and mixed powder of medicament and other additive (D50/D50)” and the “particle size distribution ratio of polymer, and mixed powder of medicament and other additive (D10/D90)” calculated from the aforementioned calculation formulas were respectively 16 and 2.2.

TABLE 2

| | Example 2 |
|---|---|
| | charge |
| | amount |
| | (g) |
| 5,5-diphenylimidazolidine-2,4-dione | 300.00 |
| lactose | 455.00 |
| corn starch | 195.00 |
| hydroxypropylcellulose | 49.95 |
| water * | 206.03 |
| total | 999.95 |

* not remaining in medicament-containing particles

### Experimental Example 1

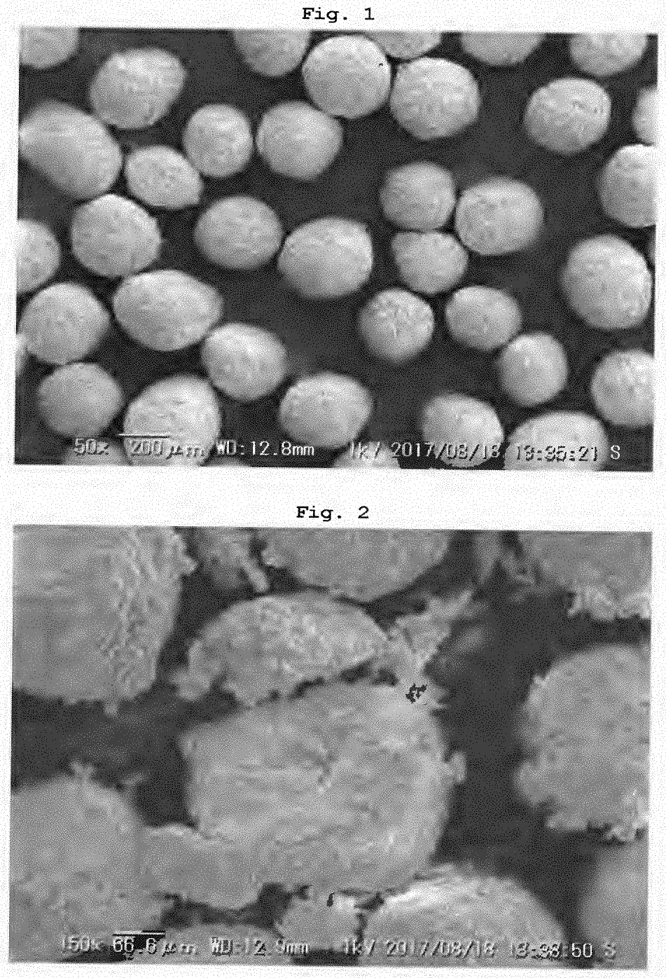

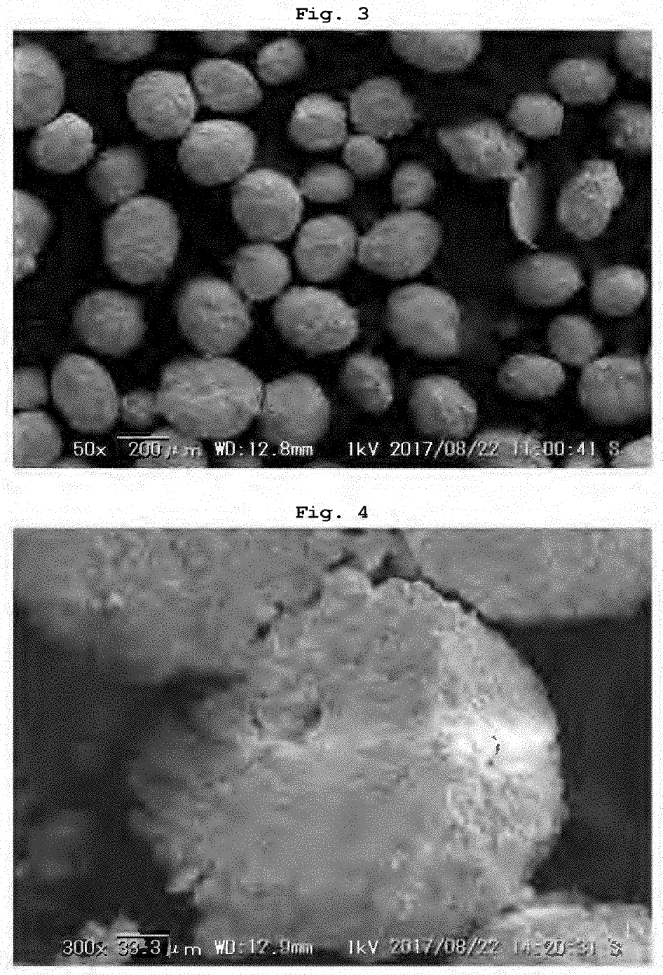

The appearance and cross-section of the medicament-containing particles of Example 1 and Example 2 were each observed using a scanning electron microscope (3D real surface view microscope, manufactured by KEYENCE CORPORATION, VE-8800) (FIG. 1-FIG. 4). According to FIG. 1-FIG. 4, the medicament-containing particles of Example 1 and Example 2 are spherical particles from the appearance, and it was confirmed from the cross-section that a hollow was present in the center of the particle.

### Experimental Example 2

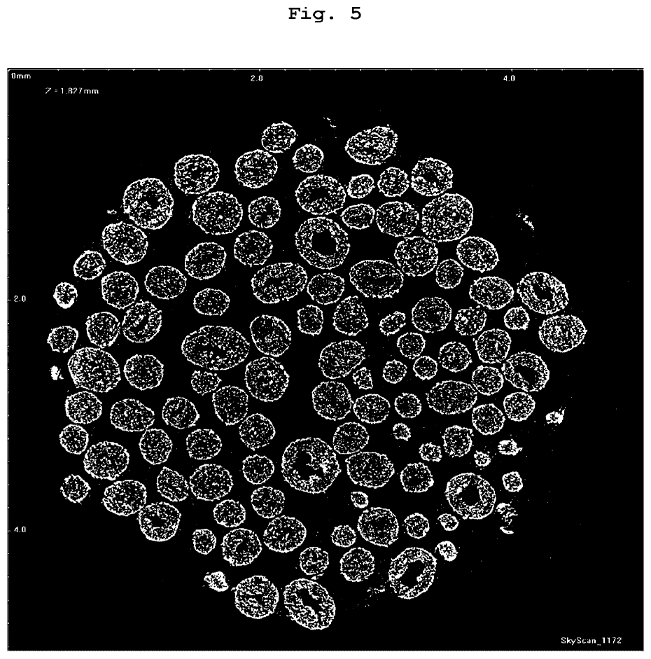

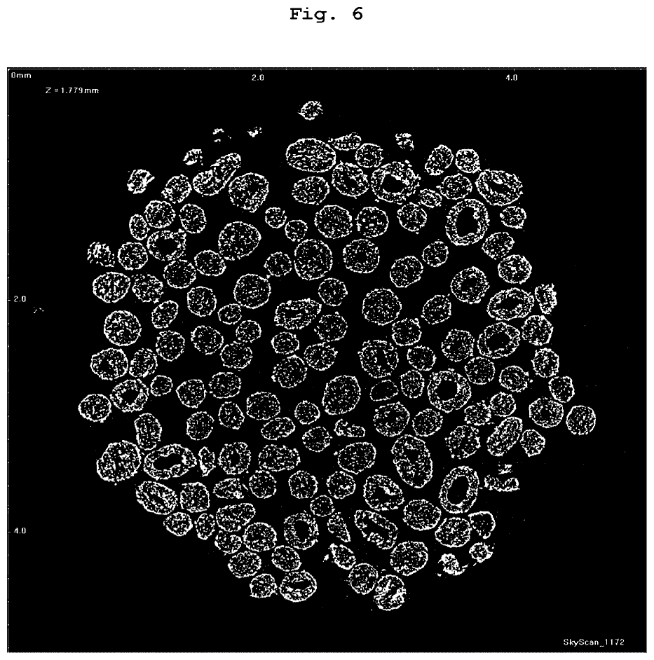

The inner structure of the medicament-containing particles produced in Example 1, Example 2 was non-destructively observed by a benchtop micro-CT (manufactured by SKYSCAN, SKYSCAN1172) (FIG. 5, FIG. 6). As a result, it was confirmed that a hollow was present in the inside of the medicament-containing particles of Example 1 and Example 2.

### Example 3

N-(4-hydroxyphenyl)acetamide and hydroxypropylcellulose indicated in the following Table 3 were charged in a rotating mixing pan (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 117.18 g of water was added dropwise for about 50 sec using a funnel, and the mixture was stirred for 29 min (stirring blade rotating speed: 3000 RPM (19.6 m/s), container rotating speed: 42 RPM (0.5 m/s)) to give medicament-containing particles in a wet powder state. The wet powdery particles were charged in a fluid bed granulator (MP-01, manufactured by POWREX CORPORATION) and dried to give the particles of Example 3.

TABLE 3

| | Example 3 |
|---|---|
| | charge |
| | amount |
| | (g) |
| N-(4-hydroxyphenyl)acetamide | 790.5 |
| hydroxypropylcellulose | 139.5 |
| water * | 117.18 |
| total | 930.00 |

* not remaining in medicament-containing particles

### Example 4

N-(4-hydroxyphenyl)acetamide and ammonicalkylmethacrylate copolymer RS indicated in the following Table 4 were charged in a rotating mixing pan (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 125.00 g of anhydrous ethanol was added dropwise for about 55 sec using a funnel, and the mixture was stirred for 1 min 45 sec (stirring blade rotating speed: 3000 RPM (19.6 m/s), container rotating speed: 42 RPM (0.5 m/s)) to give medicament-containing particles in a wet powder state. The wet powdery particles were charged in a fluid bed granulator (MP-01, manufactured by POWREX CORPORATION) and dried to give the particles of Example 4.

TABLE 4

| | Example 4 |
|---|---|
| | charge |
| | amount |
| | (g) |
| N-(4-hydroxyphenyl)acetamide | 850.00 |
| ammonioalkylmethacrylate | 150.00 |
| copolymer RS | |
| anhydrous ethanol * | 125.00 |
| total | 1000.00 |

* not remaining in medicament-containing particles

### Example 5

5,5-Diphenylimidazolidine-2,4-dione and hydroxypropylcellulose indicated in the following Table 5 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 119.85 g of water was added dropwise for 48 sec using a funnel, and the mixture was stirred for 15 min (stirring blade rotating speed: 3000 RPM (19.6 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 5.

TABLE 5

| | Example 5 |
|---|---|
| | charge |
| | amount |
| | (g) |
| 5,5-diphenylimidazolidine-2,4-dione | 851.77 |
| hydroxypropylcellulose | 150.60 |
| water * | 119.85 |
| total | 1002.37 |

* not remaining in medicament-containing particles

### Example 6

5,5-Diphenylimidazolidine-2,4-dione and hydroxypropylcellulose indicated in the following Table 6 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 1500 RPM (9.8 m/s), container rotating speed: 42 RPM (0.5 m/s)), 149.60 g of water was sprayed for 26 min using a spray device, and the mixture was stirred for 2 min (stirring blade rotating speed: 1500 RPM (9.8 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 6.

TABLE 6

| | Example 6 |
|---|---|
| | charge |
| | amount |
| | (g) |
| 5,5-diphenylimidazolidine-2,4-dione | 850.88 |
| hydroxypropylcellulose | 149.83 |
| water * | 149.60 |
| total | 1000.71 |

* not remaining in medicament-containing particles

### Example 7

5,5-Diphenylimidazolidine-2,4-dione and ammonioalkylmethacrylate copolymer RS indicated in the following Table 7 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 1500 RPM (9.8 m/s), container rotating speed: 42 RPM (0.5 m/s)), 98.50 g of anhydrous ethanol was added dropwise for 40 sec using a funnel, and the mixture was stirred for 10 min (stirring blade rotating speed: 1500 RPM (9.8 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, so manufactured by POWREX CORPORATION) and dried to give the particles of Example 7.

TABLE 7

| | Example 7 |
|---|---|
| | charge |
| | amount |
| | (g) |
| 5,5-diphenylimidazolidine-2,4-dione | 850.00 |
| ammonioalkylmethacrylate | 150.00 |
| copolymer RS | |
| anhydrous ethanol * | 98.50 |
| total | 1000.00 |

* not remaining in medicament-containing particles

### Example 8

5,5-Diphenylimidazolidine-2,4-dione and ammonioalkylmethacrylate copolymer RS indicated in the following Table 8 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 1500 RPM (9.8 m/s), container rotating speed: 42 RPM (0.5 m/s)), 139.88 g of anhydrous ethanol was sprayed for 23 min using a spray device, and the mixture was stirred for 2 min (stirring blade rotating speed: 1500 RPM (9.8 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 8.

TABLE 8

| | Example 8 |
|---|---|
| | charge |
| | amount |
| | (g) |
| 3,5-diphenylimidazolidine-2,4-dione | 850.00 |
| ammonioalkylmethacrylate | 150.00 |
| copolymer RS | |
| anhydrous ethanol * | 139.88 |
| total | 1000.00 |

* not remaining in medicament-containing particles

### Example 9

Benzo[d]isoxazol-3-ylmethanesulfonamide and hydroxypropylcellulose indicated in the following Table 9 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 105.74 g of water was added dropwise for 45 sec using a funnel, and the mixture was stirred for 25 min (stirring blade rotating speed: 3000 RPM (19.6 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 9.

TABLE 9

| | Example 9 |
|---|---|
| | charge |
| | amount |
| | (g) |
| benzo[d]isoxazol-3-ylmethanesulfonamide | 848.97 |
| hydroxypropylcellulose | 150.12 |
| water | 105.74 |
| total | 999.09 |

* not remaining in medicament-containing particles

### Example 10

Benzo[d]isoxazol-3-ylmethanesulfonamide and hydroxypropylcellulose indicated in the following Table 10 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 135.88 g of water was sprayed for 22 min 30 sec using a spray device, and the mixture was stirred for 2 min (stirring blade rotating speed: 3000 RPM (19.6 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 10.

TABLE 10

| | Example 10 |
|---|---|
| | charge |
| | amount |
| | (g) |
| benzo[d]isoxazol-3-ylmethanesulfonamide | 847.19 |
| hydroxypropylcellulose | 150.20 |
| water * | 135.38 |
| total | 997.39 |

* not remaining in medicament-containing particles

### Example 11

Benzo[d]isoxazol-3-ylmethanesulfonamide and ammonioalkylmethacrylate copolymer RS indicated in the following Table 11 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 103.84 g of anhydrous ethanol was added dropwise for 40 sec using a funnel, and the mixture was stirred for 8 min (stirring blade rotating speed: 3000RPM (19.6 m/s), container rotating speed: 42 RPM (C.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 11.

TABLE 11

| | Example 11 |
|---|---|
| | charge |
| | amount |
| | (g) |
| benzo[d]isoxazol-3-ylmethanesulfonamide | 849.42 |
| ammonioalkylmethacrylate copolymer RS | 150.21 |
| anhydrous ethanol * | 103.84 |
| total | 999.63 |

* not remaining in medicament-containing particles

### Example 12

Benzo[d]isoxazol-3-ylmethanesulfonamide and ammonioalkylmethacrylate copolymer RS indicated in the following Table 12 were charged in a rotating mixing pan equipped with a scraper (Eirich CleanLine C5, manufactured by NIPPON EIRICH CO., LTD.) and the mixture was premixed for 2 min (stirring blade rotating speed: 1500 RPM (9.8 m/s), container rotating speed: 42 RPM (0.5 m/s)). Successively, with stirring (stirring blade rotating speed: 3000 RPM (19.6 m/s), container rotating speed: 42 RPM (0.5 m/s)), 133.51 g of anhydrous ethanol was sprayed for 23 min using a spray device, and the mixture was stirred for 3 min (stirring blade rotating speed: 3000 RPM (19.6 m/s), container rotating speed: 42 RPM (0.5 m/s)) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Example 12.

TABLE 12

| | Example 12 |
|---|---|
| | charge |
| | amount |
| | (g) |
| benzo[d]isoxazol-3-ylmethanesulfonamide | 849.79 |
| ammonioalkylmethacrylate copolymer RS | 149.86 |
| anhydrous ethanol * | 133.51 |
| total | 999.65 |

* not remaining in medicament-containing particles

### Example 13

5,5Diphenylimidazolidine-2,4-dione and hydroxypropylcellulose indicated in the following Table 13 were charged in a rotating mixing pan (Eirich CleanLine C50, manufactured by NIPPON EIRICH CO., LTD.) and the mixture was premixed for 2 min (stirring blade rotating speed: 1400 RPM (20.5 m/s), container rotating speed: 25 RPM (0.78 m/s)). Successively, with stirring (stirring blade rotating speed: 1400 RPM (20.5 m/s), container rotating speed: 25 RPM (0.78 m/s)), 1200 g of water was added dropwise for 1 min using a funnel, and the mixture was stirred for 30 min (stirring blade rotating speed: 1400 RPM (20.5 m/s), container rotating speed: 25 RPM (0.78 m/s)) to give medicament-containing particles in a wet powder state. The wet powdery particles were air dried to give the particles of Example 13.

TABLE 13

| | Example 13 |
|---|---|
| | charge |
| | amount |
| | (g) |
| 5,5-diphenylimidazolidine-2,4-dione | 8500 |
| hydroxypropylcellulose | 1500 |
| water * | 1200 |
| total | 10000 |

* not remaining in medicament-containing particles

### Example 14

5,5-Diphenylimidazolidine-2,4-dione and hydroxypropylcellulose indicated in the following Table 14 were charged in a rotating mixing pan (Eirich CleanLine C50, manufactured by NIPPON EIRICH CO., LTD.) and the mixture was premixed for 2 min (stirring blade rotating speed: 1400 RPM (20.5 m/s), container rotating speed: 25 RPM (0.78 m/s)). Successively, with stirring (stirring blade rotating speed: 1400 RPM (20.5 m/s), container rotating speed: 25 RPM (0.78 m/s)), 1440 g of water was sprayed for about 45 min using a spray device, and the mixture was stirred for 15 sec (stirring blade rotating speed: 1400 RPM (20.5 m/s), container rotating speed: 25 RPM (0.78 m/s)) to give medicament-containing particles in a wet powder state. The wet powdery particles were air dried to give the particles of Example 14.

TABLE 14

| | Example 14 |
|---|---|
| | charge |
| | amount |
| | (g) |
| 5,5-diphenylimidazolidine-2,4-dione | 8500 |
| hydroxypropylcellulose | 1500 |
| water * | 1440 |
| total | 10000 |

* not remaining in medicament-containing particles

### Comparative Example 1

5,5-Diphenylimidazolidine-2,4-dione and hydroxypropylcellulose indicated in the following Table 15 were charged in a high-speed mixer granulator (vertical granulator VG05, manufactured by POWREX CORPORATION) and the mixture was premixed for 2 min (blade rotating speed: 400 RPM, cross screw rotating speed: 3000 RPM). Successively, with stirring (blade rotating speed: 400 RPM, cross screw rotating speed, 3000 RPM), 105.00 g of water was sprayed for about 17 min 30 sec using a spray device, and the mixture was stirred for 2 min (blade rotating speed: 400 RPM, cross screw rotating speed: 3000 RPM) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Comparative Example 1.

TABLE 15

| | Comparative |
|---|---|
| | Example 1 |
| | charge |
| | amount |
| | (g) |
| 5,5-diphenylimidazolidine-2,4-dione | 595.00 |
| hydroxypropylcellulose | 105.00 |
| water * | 105.00 |
| total | 700.00 |

* not remaining in medicament-containing particles

### Comparative Example 2

5,5-Diphenylimidazolidine-2,4-dione and ammonioalkylmethacrylate copolymer RS indicated in the following Table 16 were charged in a high-speed mixer granulator (vertical granulator VG05, manufactured by POWREX CORPORATION) and the mixture was premixed for 2 min (blade rotating speed: 400 RPM, cross screw rotating speed: 3000 RPM). Successively, with stirring (blade rotating speed: 400 RPM, cross screw rotating speed, 3000 RPM), 120.00 g of anhydrous ethanol was sprayed for about 10 min using a spray device, and the mixture was stirred for 2 min (blade rotating speed: 400 RPM, cross screw rotating speed: 3000 RPM) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Comparative Example 2.

TABLE 16

| | Comparative |
|---|---|
| | Example 2 |
| | charge |
| | amount |
| | (g) |
| 5,5-diphenylimidazolidine-2,4-dione | 595.00 |
| ammonioalkylmethacrylate | 105.00 |
| copolymer RS | |
| anyhdrous ethanol * | 120.00 |
| total | 700.00 |

* not remaining in medicament-containing particles

### Comparative Example 3

Benzo[d]isoxazol-3-ylmethanesulfonamide and hydroxypropylcellulose indicated in the following Table 17 were charged in a high-speed mixer granulator (vertical granulator VG05, manufactured by POWREX CORPORATION) and the mixture was premixed for 2 min (blade rotating speed: 400 RPM, cross screw rotating speed: 3000 RPM). Successively, with stirring (blade rotating speed: 400 RPM, cross screw rotating speed, 3000 RPM), 100.00 g of water was sprayed for about 12 min 30 sec using a spray device, and the mixture was stirred for 2 min (blade rotating speed: 400 RPM, cross screw rotating speed: 3000 RPM) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Comparative Example 3.

TABLE 17

| | Comparative |
|---|---|
| | Example 3 |
| | charge |
| | amount |
| | (g) |
| benzo[d]isoxazel-3-ylmethanesulfonamide | 595.00 |
| hydroxypropyleellulose | 105.00 |
| water * | 100.00 |
| total | 700.00 |

* not remaining in medicament-containing particles

### Comparative Example 4

Benzo[d]isoxazol-3-ylmethanesulfonamide and ammonioalkylmethacrylate copolymer RS indicated in the following Table 18 were charged in a high-speed mixer granulator (vertical granulator VG05, manufactured by POWREX CORPORATION) and the mixture was premixed for 2 min (blade rotating speed: 400 RPM, cross screw rotating speed: 3000 RPM). Successively, with stirring (blade rotating speed: 400 RPM, cross screw rotating speed, 3000 RPM), 170.00 g of anhydrous ethanol was sprayed for about 28 min 20 sec using a spray device, and the mixture was stirred for 2 min (blade rotating speed: 400 RPM, cross screw rotating speed: 3000 RPM) to give medicament-containing particles in a wet powder state. No scraping operation was performed on the inside of the container when producing the medicament-containing particles. The wet powdery particles were charged in a fluid bed granulator (MP-01, manufactured by POWREX CORPORATION) and dried to give the particles of Comparative Example 4.

TABLE 18

| | Comparative |
|---|---|
| | Example 4 |
| | charge |
| | amount |
| | (g) |
| benzo[d]isoxazol-3-ylmethanesulfonamide | 595.00 |
| ammonioalkylmethacrylate copolymer RS | 105.00 |
| anhydrous ethanol * | 170.00 |
| total | 700.00 |

* not remaining in medicament-containing particles

### Experimental Example 3

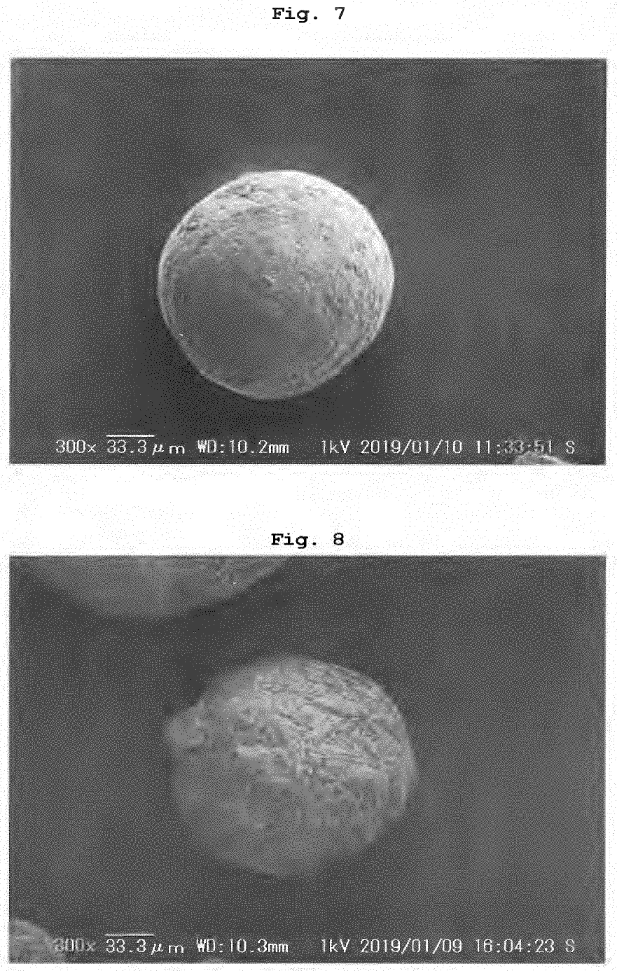

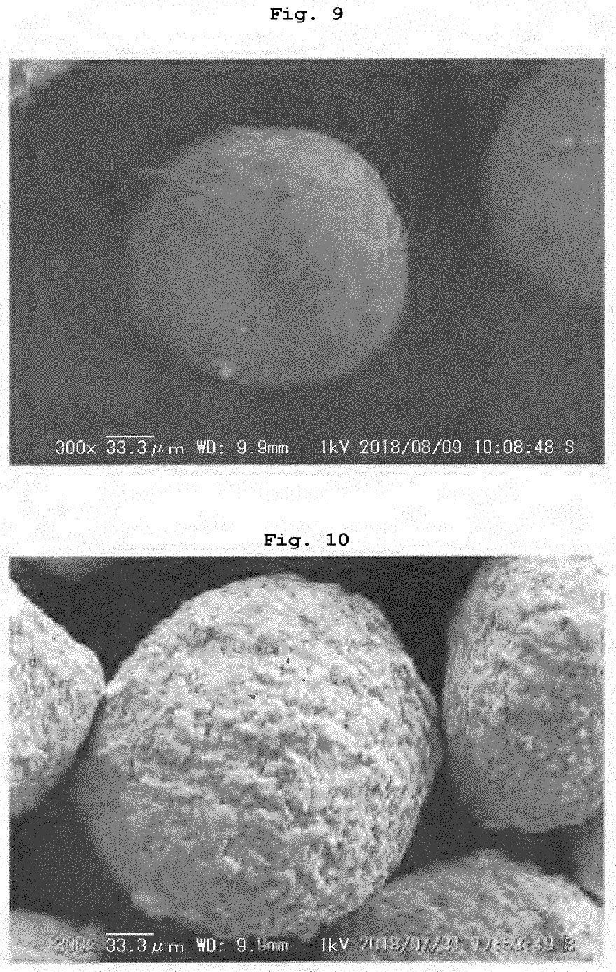

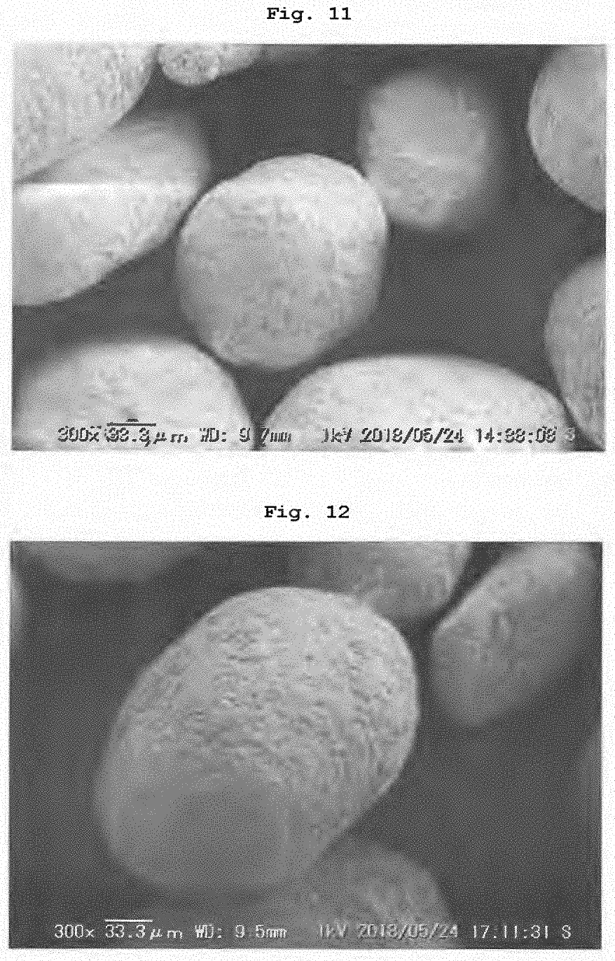

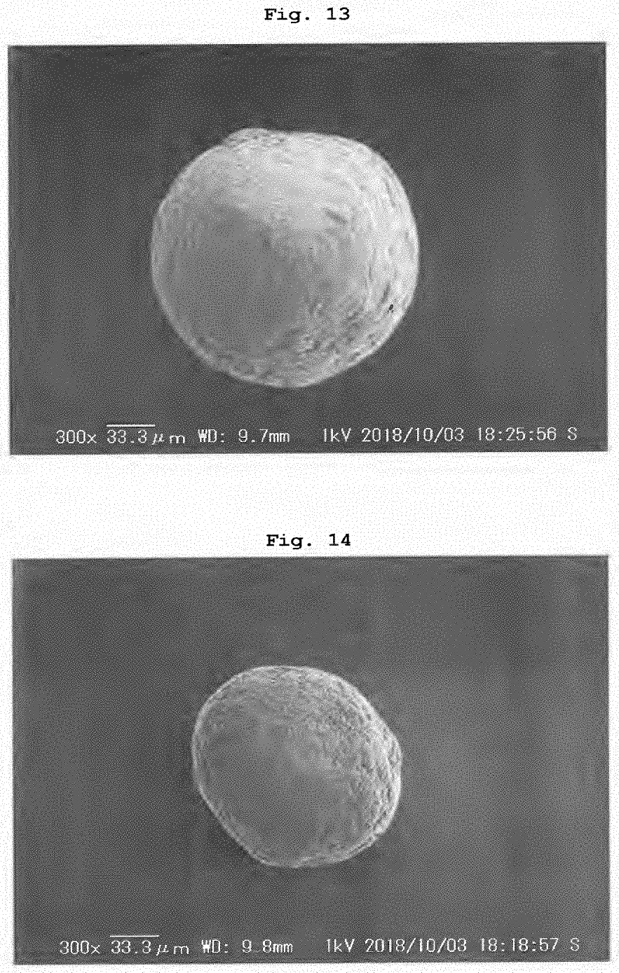

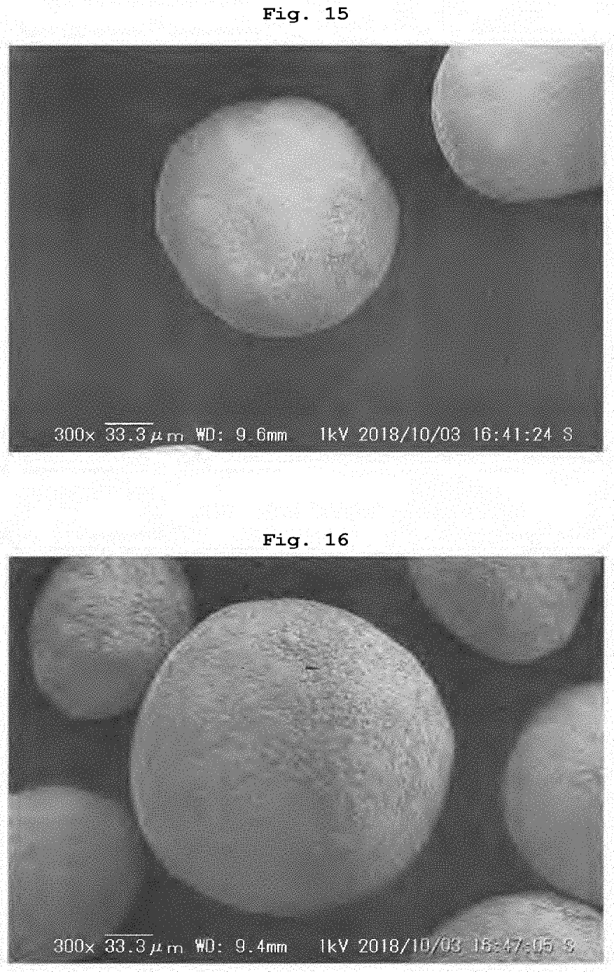

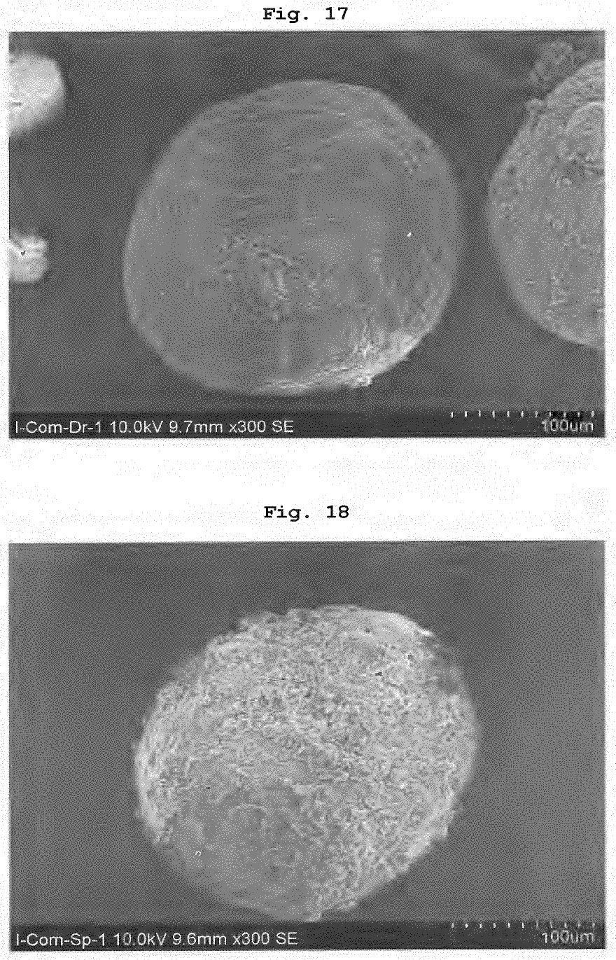

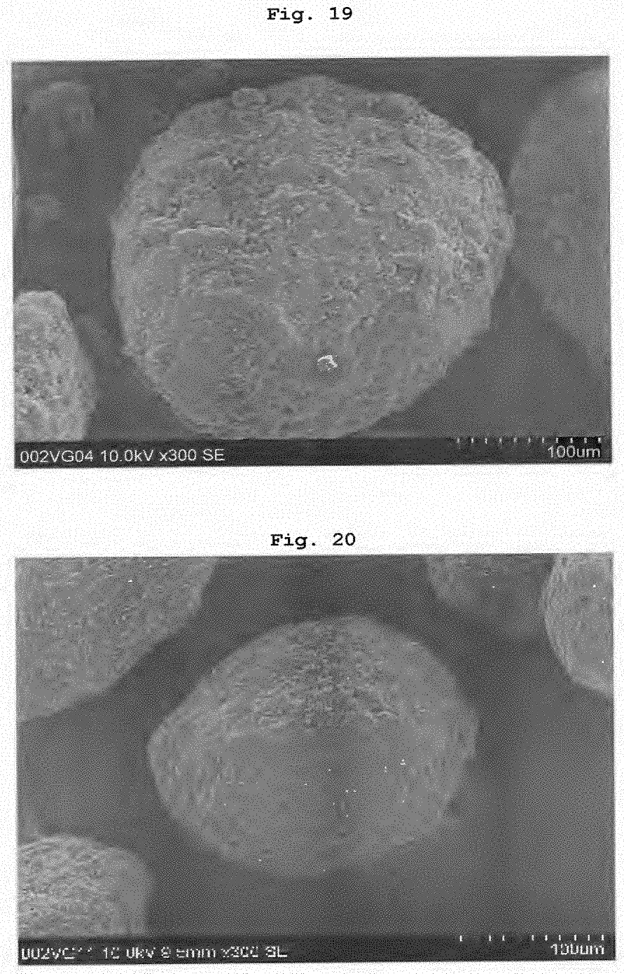

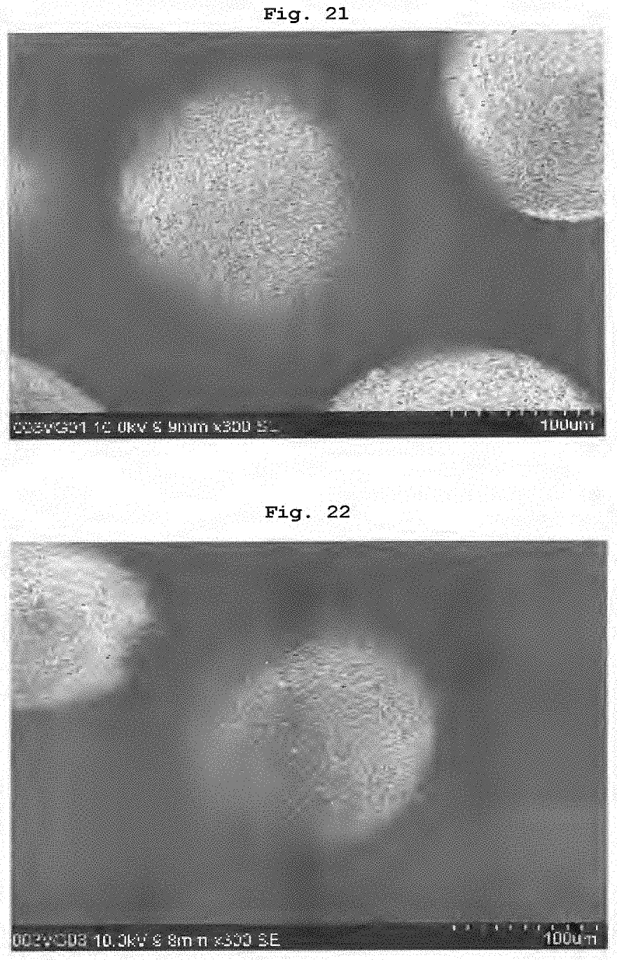

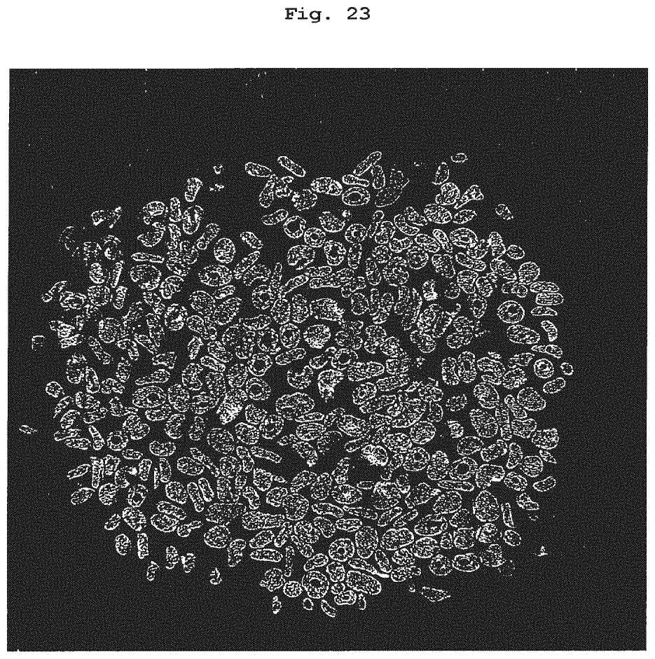

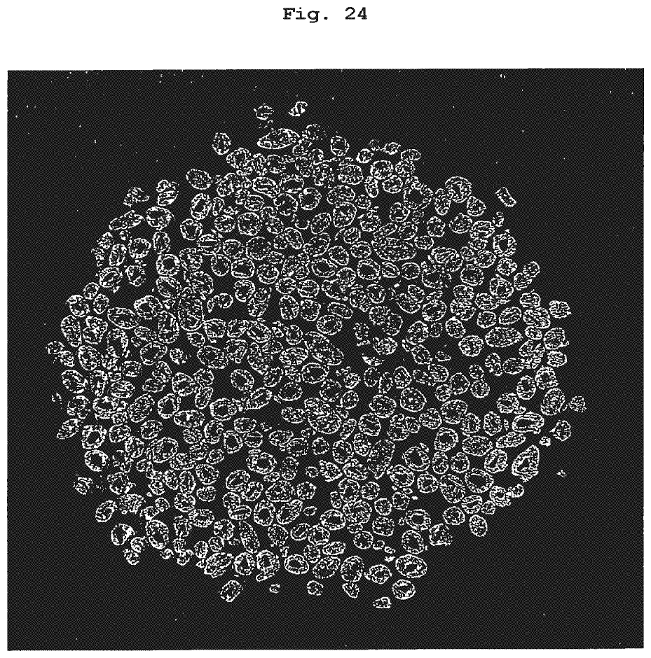

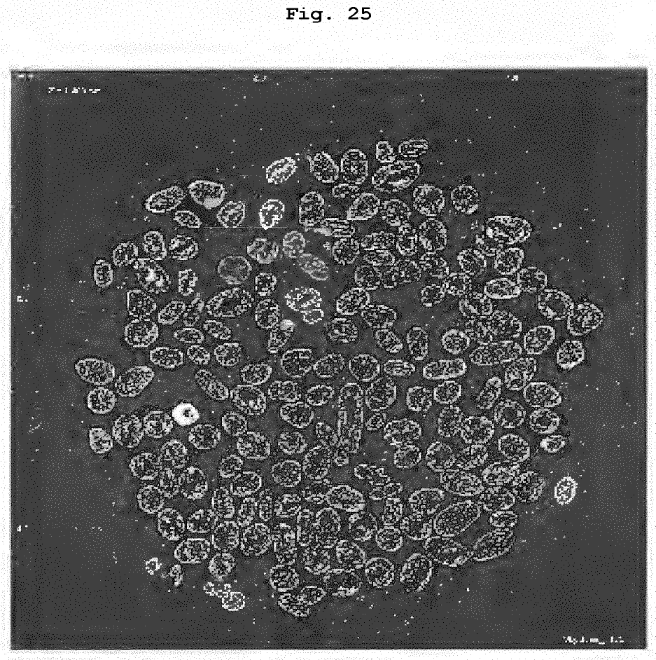

The appearance of the medicament-containing particles of Examples 3-14 and Comparative Examples 1-4 was each observed using a scanning electron microscope (3D real surface view microscope, manufactured by KEYENCE CORPORATION, VE-8800) (FIG. 7-FIG. 22). According to FIG. 7-FIG. 22, it was confirmed that the medicament-containing particles of Examples 3-14 and Comparative Examples 1-4 were spherical particles from the appearance.

### Experimental Example 4

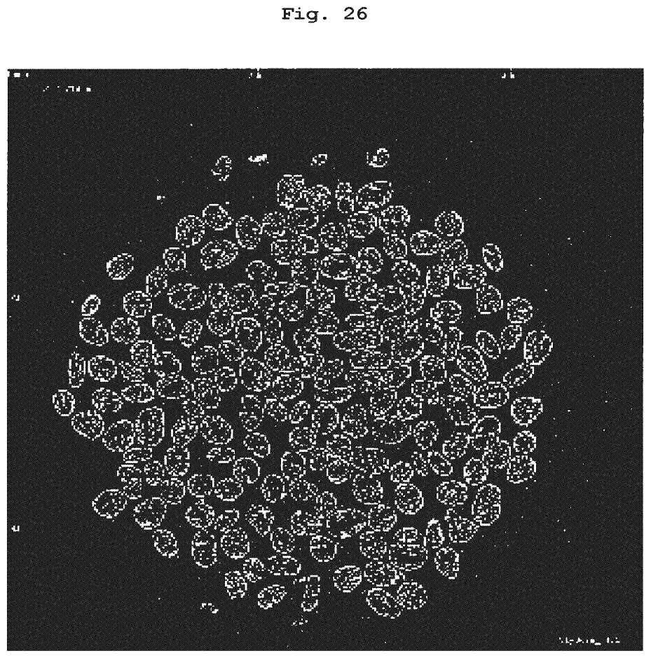

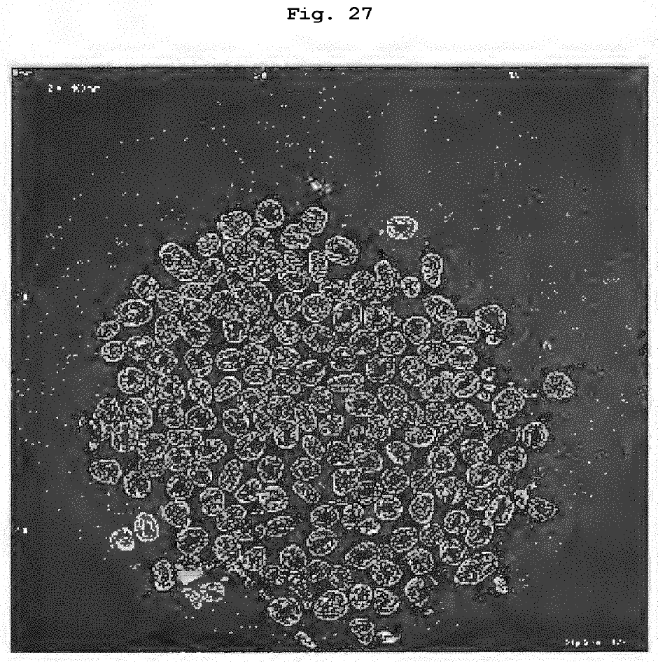

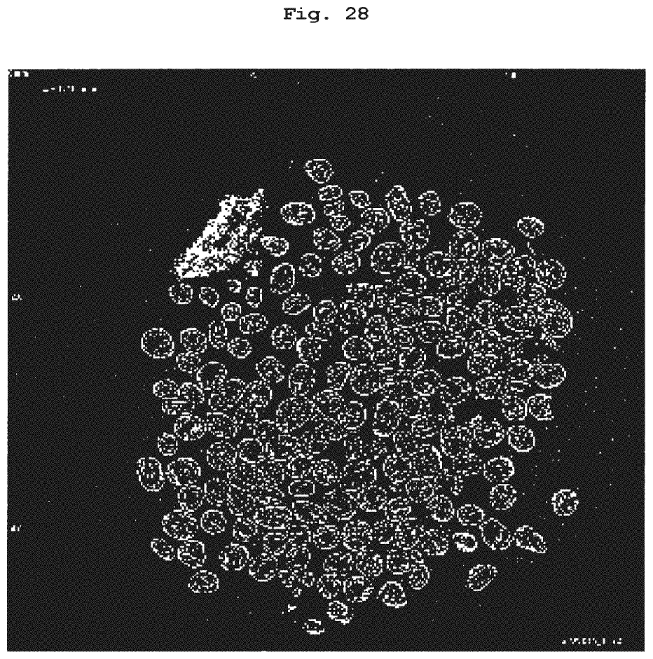

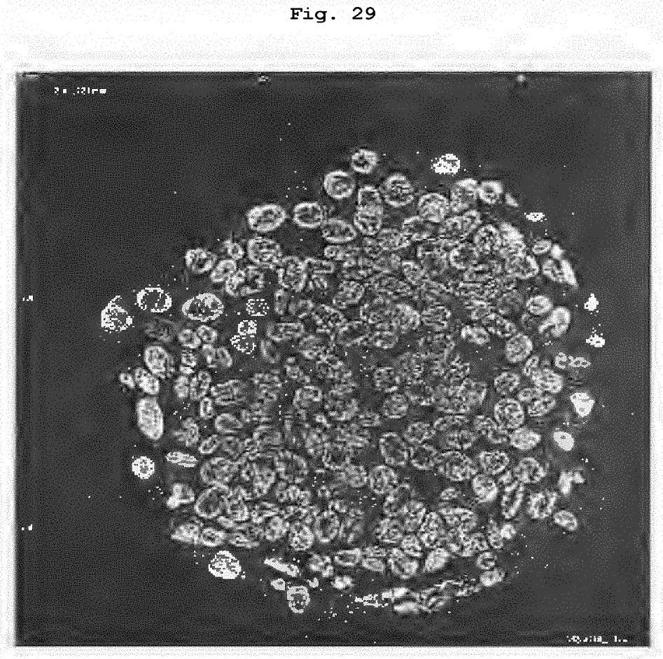

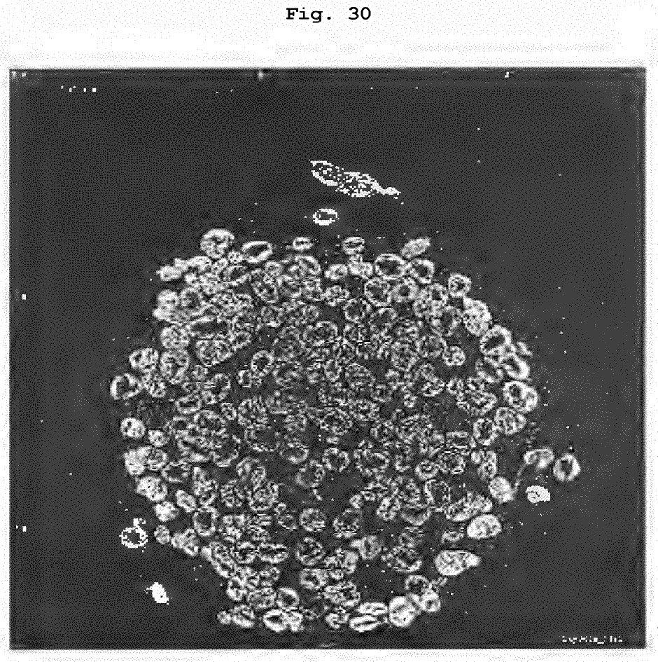

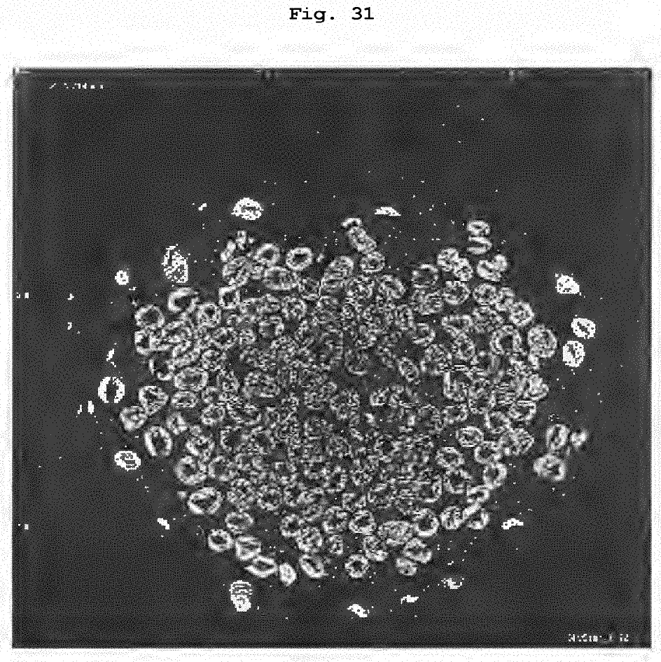

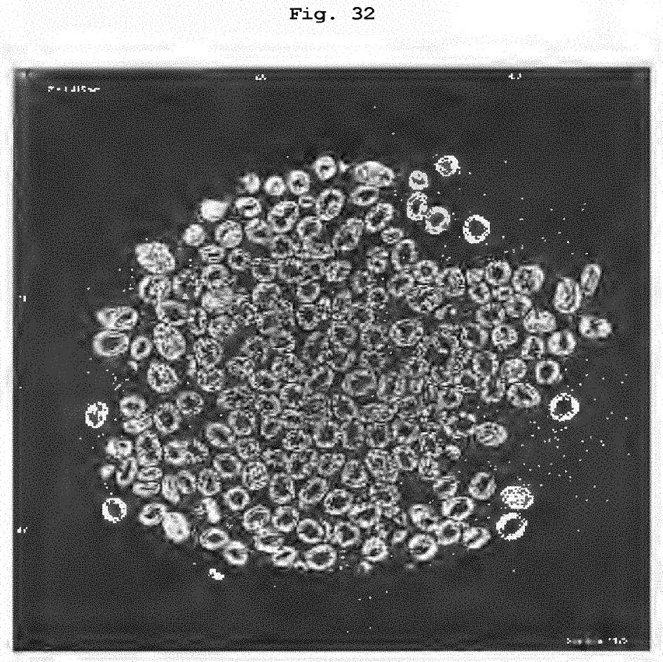

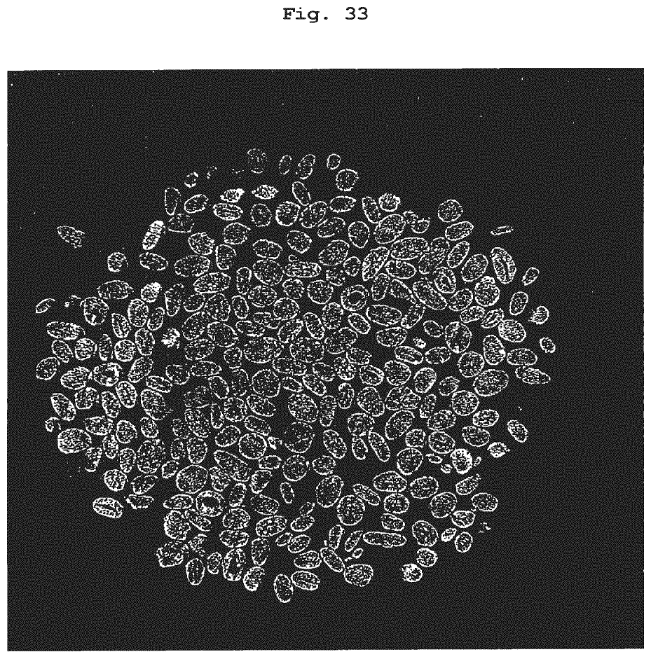

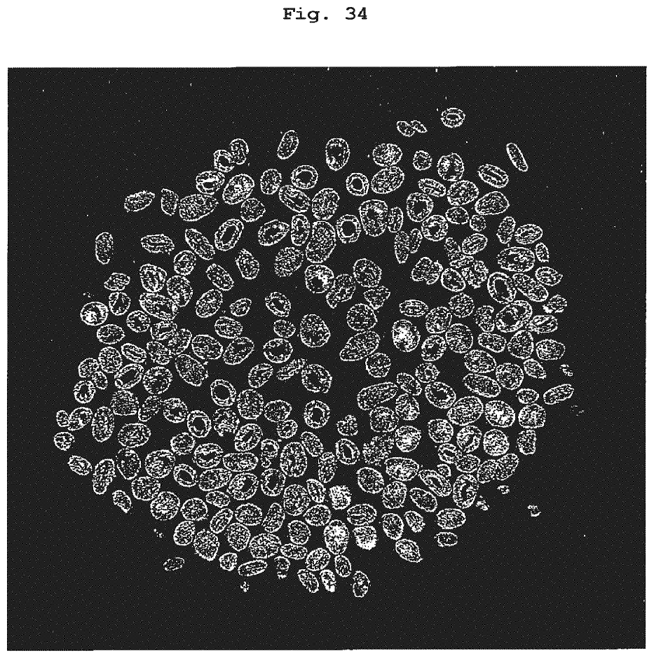

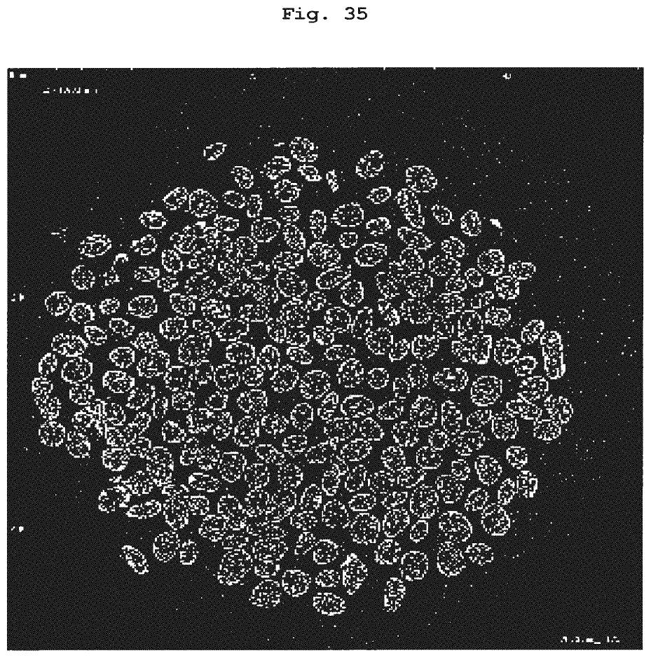

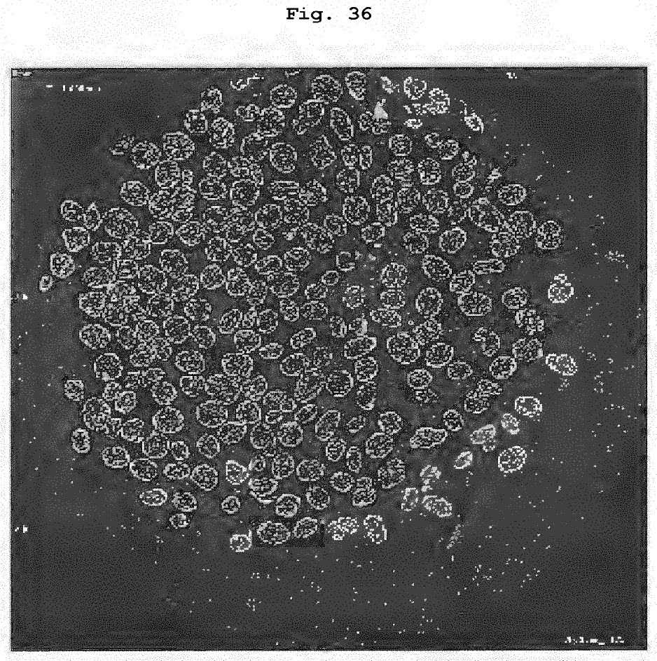

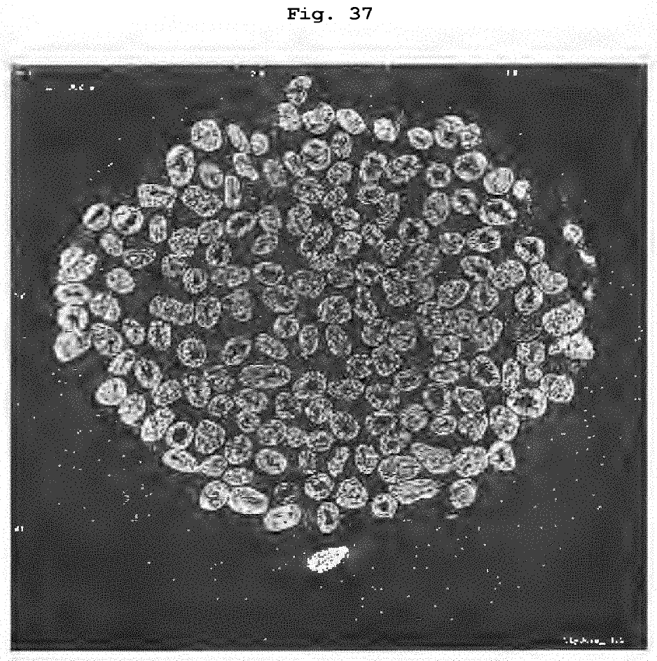

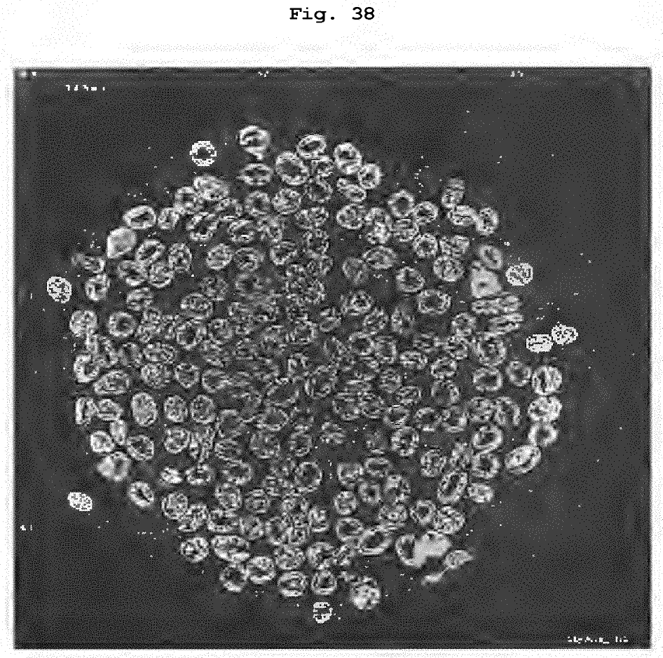

The inner structure of the medicament-containing particles produced in Examples 3-14 and Comparative Examples 1-4 was non-destructively observed by a benchtop micro-CT (manufactured by SKYSCAN, SKYSCAN1172) (FIG. 23-FIG. 38). As a result, it was confirmed that a hollow was present in the inside of the medicament-containing particles of Examples 3-14 and Comparative Examples 1-4.

### Experimental Example 5

After producing the medicament-containing particles of Examples 5-14 and Comparative Examples 1-4, all medicament-containing particles not adhered to the container were recovered, and the recovered product was sieved with a 500 μm sieve. A ratio to the charge amount of the 500 μm sieving through product (good quality) was calculated (Table 19, Table 20). According to Table 19, Examples 5, 7, 9, 11, 13 and 14 showed a high yield permitting automatic granulation and, according to Table 20, it was confirmed that the 500 μm sieving through products (good quality) of Examples 6, 8, 10 and 12 showed a higher ratio than the 500 μm sieving through products (good quality) obtained in Comparative Examples 1-4. In (Table 20), the same components are described in the upper panel and the lower panel to allow for comparison.

TABLE 19

| Example | Example | Example | Example | Example | Example |
|---|---|---|---|---|---|
| 5 | 7 | 9 | 11 | 13 | 14 |
| 81.5% | 84.8% | 68.6% | 81.9% | 88.0% | 85.1% |

TABLE 20

| Example 6 | Example 8 | Example 10 | Example 12 |
|---|---|---|---|
| 88.3% | 85.9% | 72.6% | 76.4% |
| Comparative | Comparative | Comparative | Comparative |
| Example 1 | Example 2 | Example 3 | Example 4 |
| 60.7% | 74.9% | 57.6% | 69.4% |

### INDUSTRIAL APPLICABILITY

According to the present invention, a spherical, medicament-containing particle having sufficient strength of a level that facilitates processing such as compression, coating and the like, and having a hollow structure with a pharmaceutically useful and desired function can be provided in a short time and conveniently compared to conventional production methods. In addition, the present invention makes it possible to decrease adhesion of the medicament-containing particles to the inner side wall of a device, and drastically improve the yield in the production step of the medicament-containing particle.

This application is based on a patent application No. 2018-026095 filed in Japan, the contents of which are incorporated in full herein.

1-12. (canceled)

13. A method for producing a hollow particle comprising a hollow and a shell comprising a medicament and a polymer, the method comprising adding a polymer and a solvent capable of dissolving the polymer to a powder comprising a medicament in a rotating mixing pan, and subsequently granulating the mixture.

14. The method of claim 13, wherein a volume ratio of the hollow relative to the whole particle of the medicament-containing particle is 1%-50%.

15. The method of claim 13 thickness of the particle is not less than 10 μm.

16. The method of claim 13, wherein the polymer used as a starting material has an average particle size prior of not less than 5-fold that of the medicament containing powder.

17. The method of claim 16, wherein the polymer and solvent are added to medicament containing power while rotating the container and a stirring blade by using the rotating mixing pan.

18. The method of claim 13, wherein the polymer is mixed as a powder with the medicament and the solvent.

19. The method of claim 13, wherein the polymer is added to the medicament containing powder followed by the addition of the solvent capable of dissolving the polymer.

20. The method of claim 13, wherein the solvent capable of dissolving the polymer is added to medicament containing powder followed by the addition of the polymer.

21. The method of claim 13, wherein the rotating mixing pan has a scraper.

22. The method of claim 13, wherein the solvent capable of dissolving the polymer is added dropwise or added by spraying.

23. The production method according to claim 13, wherein the solvent capable of dissolving the polymer are added dropwise.

24. The production method according to claim 13, wherein the solvent capable of dissolving the polymer are added by spraying.

25. The method of claim 13, wherein a volume ratio of the hollow relative to the whole particle of the medicament-containing particle is 1%-50%, and a shell thickness is not less than 15 μm.

26. The of method of claim 13, wherein the polymer is one or more kinds selected from the group consisting of a water-soluble polymer, a water-insoluble polymer, an enteric polymer, a gastric soluble polymer and a biodegradable polymer.

27. The method of claim 13, wherein the polymer is at least one water-soluble polymer selected from the group consisting of methylcellulose, hydroxypropylcellulose, hydroxypropylmethylcellulose, hydroxyethyl cellulose, hydroxymethylcellulose, carboxymethylcellulose, polyvinylpyrrolidone, polyvinyl alcohol, copolyvidone, polyethylene glycol, polyvinyl alcohol-acrylic acid-methyl methacrylate copolymer, vinyl acetate-vinylpyrrolidone copolymer, polyvinyl alcohol-polyethylene glycol-graft copolymer, pregelatinized starch, dextrin, dextran, pullulan, alginic acid, gelatin, and pectin.

28. The method of claim 13, wherein the polymer is at least one water-insoluble polymer selected from the group consisting of ethylcellulose, acetyl cellulose, aminoalkylmethacrylate copolymer RS, ethyl acrylate-methyl methacrylate copolymer dispersion, and vinyl acetate resin.

29. The method of claim 13, wherein the polymer is at least one enteric polymer selected from the group consisting of hydroxypropylmethylcellulose acetate succinate, hydroxypropylmethylcellulose phthalate, methacrylic acid copolymer L, methacrylic acid copolymer LD, dried methacrylic acid copolymer LD, methacrylic acid copolymer S, and methacrylic acid-n-butyl acrylate copolymer.

30. The method claim 13, wherein the shell further comprises an additive.

31. The method claim 30, wherein the additive is at least one selected from the group consisting of filler, binder, sweetening agent, corrigent, smell masking agent, flavor, fluidizer, antistatic agent, colorant, disintegrant, lubricant, plasticizer, anticoagulant and coating agent.

32. A pharmaceutical composition comprising the medicament-containing particle produced by the production method according to claim 13.

33. A method for producing a tablet comprising a step of tableting the medicament-containing particle produced by the production method according to claim 13.
